(19) United States
(12) Patent Application Publication
(10) Pub. No.: US 2009/0306499 A1
(43) Pub. Date: Dec. 10, 2009

(54) SELF-DETECTING KINEMATIC CLAMP ASSEMBLY

(75) Inventors: Robert Van Vorhis, Davis, CA (US); Amardeep Singh Dugal, Davie, FL (US); Timothy W. Perez, Fort Lauderdale, FL (US)

Correspondence Address: PROSKAUER ROSE LLP, ONE INTERNATIONAL PLACE, BOSTON, MA, 02110, US

(73) Assignee: MAKO Surgical Corp., Fort Lauderdale, FL (US)

(21) Appl. No.: 12/479,510

(22) Filed: Jun. 5, 2009

Related U.S. Application Data
(60) Provisional application No. 61/131,487, filed on Jun. 9, 2008.

(51) Int. Cl. A61B 5/05; A61B 19/00

(52) U.S. Cl. 600/426; 606/130

(57) ABSTRACT

Described are computer-based surgical methods and apparatuses for a self-detecting kinematic assembly. The self-detecting kinematic clamp assembly detects and indicates a degradation in pose. The self-detecting kinematic clamp assembly includes a tracking element for tracking a pose of the kinematic clamp assembly. The self-detecting kinematic clamp assembly includes a base portion comprising a fixation device that is attachable to a rigid body, and a top portion connected to the tracking element, the top portion being removably connected to the base portion. The self-detecting kinematic clamp assembly includes a detection mechanism, the detection mechanism including three or more contact points between the base potion and top portion and a mechanism that detects movement between one or more of the three or more contact points.

### CROSS REFERENCES TO RELATED APPLICATIONS

This application relates to and is assigned to the same entity as the co-pending application identified by Attorney Docket No. M077, entitled “Fifth marker and kinematic clamp,” U.S. Provisional Patent Application No. 61/131,487, filed on Jun. 9, 2008, the disclosure of which is hereby incorporated herein by reference in its entirety.

### FIELD OF THE INVENTION

The present invention relates generally to surgical systems and methods and, more particularly, to self-detecting kinematic clamp assemblies.

### BACKGROUND

Orthopedic joint replacement surgery may involve arthroplasty of a knee, hip, or other joint (e.g., shoulder, elbow, wrist, ankle, finger, etc.). During joint replacement surgery, a surgeon typically removes diseased bone from the joint and replaces the resected bone with prosthetic implant components. Challenges of joint replacement surgery include determining the appropriate position for implant components within the joint relative to the bone and other implant components and accurately cutting and reshaping bone to precisely correspond to the planned placement of the implant components. Inaccurate positioning of implants may compromise joint performance and reduce implant life.

Surgical systems can include a haptic device, which is a surgical device configured to be manipulated by a user to move a surgical tool to perform a procedure on a patient, such as, for example, a joint replacement procedure in which a surgeon manipulates the haptic device to sculpt bone and installs an implant component on the sculpted bone. The haptic device, coupled with computer aided surgery (CAS), can enable a surgeon to actively and accurately control surgical actions and delivery of localized therapies. A computing system implements control parameters for controlling the haptic device based on, for example, a relationship between an anatomy of the patient and a position, orientation, velocity, and/or acceleration of a portion of the haptic device, such as a surgical cutting tool coupled to the haptic device. A three-dimensional model of an area of a patient upon which a surgical procedure is to be performed is modeled using software techniques. The software model is used to generate a surgical plan, including, for example, resecting bone and/or cartilage (e.g., using the surgical cutting tool) and inserting implant components. A tracking system can be used to calculate a pose (i.e., position and/or orientation) of the portion of the patient's anatomy that corresponds to the three-dimensional model. Using pose data from the tracking system, a surgeon can use the surgical system to register (or map or associate) coordinates in one space (e.g., model or image space) to those in another (e.g., physical space) to achieve spatial alignment or correspondence (e.g., using a coordinate transformation process as is well known).

For example, U.S. patent application Ser. No. 11/357,197 (U.S. Pub. No. 2006/0142657), which is hereby incorporated by reference herein in its entirety, describes that objects in physical space (e.g., anatomy, surgical tools, etc.) may be registered to any suitable coordinate system, such as a coordinate system being used by a process running on a computer associated with a surgical system. For example, utilizing object pose data captured by a tracking system, the surgical system can associate the physical anatomy and the surgical tool (and/or the haptic device) with a representation of the anatomy (such as a computer-generated three-dimensional model or image of the anatomy). Based on the tracked object and registration data, the surgical system may determine, for example, (a) a spatial relationship between the image of the anatomy and the relevant physical anatomy and (b) a spatial relationship between the relevant physical anatomy and the surgical tool so that the computing system can superimpose (and continually update) a virtual representation of the tool on the image of the anatomy, where the relationship between the virtual representation and the image is substantially identical to the relationship between the surgical tool and the physical anatomy. Additionally, by tracking not only the tool but also the relevant anatomy, the surgical system can compensate for movement of the relevant anatomy during the surgical procedure (e. g., by adjusting a virtual object that defines a surgical cutting boundary in response to the detected movement of the anatomy).

The tracking system (e.g., a non-mechanical tracking system or a mechanical tracking system) enables the surgical system to continually determine (or track) a pose of the relevant anatomy of the patient. Non-mechanical tracking systems often include a detection device adapted to locate in a predefined coordinate space specially recognizable trackable elements (e.g., active or passive markers) that are detectable by the detection device and that are configured to be attached to the tracked object (e.g., affixed directly to the tracked object or to a tracking array that is affixed to the tracked object). However, after registration, if the physical relationship between the trackable elements and the relevant anatomy changes, and the occurrence of the change is not known or compensated for, the pose of the relevant anatomy will be incorrectly predicted based on the changed location of the trackable elements. This “registration degradation” can occur, for example, from the trackable elements being inadvertently bumped by a member of the surgical team, from interference, or from non-rigid attachment.

Surgical systems can verify the registration or calibration of a surgical device. For example, U.S. patent application Ser. No. 11/750,807 (U.S. Pub. No. 2008/0004633), which is hereby incorporated by reference herein in its entirety, describes identifying at least one interface on the anatomy and determining a position of a checkpoint of the interface in a coordinate frame of reference, such as by digitizing the checkpoint when a tracked probe is in contact with the interface. The interface may be, for example, a painted portion on a bone of the patient, a divot made in the bone, or a mechanical interface disposed on the bone. During the surgical procedure, the checkpoint enables a user to verify that the surgical system is properly configured. For example, the user can touch the tip of a tracked probe or tool to the interface to confirm that the tracking system is properly configured (e.g., the trackable elements are not occluded and are still properly aligned relative to the anatomy and/or the haptic device, etc.), that the tool is correctly installed (e.g., properly seated, shaft not bent, etc.), and/or that any other object is properly mounted, installed, calibrated, registered, and the like.

In this manner, the checkpoint enables the surgical system to confirm that all elements involved in relating the tip of the surgical tool to the anatomy of the patient remain in registration or calibration and that the trackable elements are updating properly. Because the user must proactively verify that the surgical system is properly configured, however, the current and correct registration can not always be guaranteed. For example, if registration degradation occurs immediately after the user verifies the registration via the checkpoint, the surgical system will be improperly configured unbeknownst to the user, which can lead to the surgical plan being carried out improperly (e.g., performing improper resections). Similar problems can occur with mechanical tracking systems.

In view of the foregoing, a need exists for surgical methods and devices which can overcome the aforementioned problems so as to enable CAS to be carried out when degradation of the registration occurs between the patient's physical anatomy and the representation of the physical anatomy in the CAS model by immediately detecting the registration degradation without requiring proactive steps to be taken on the part of the user or more importantly, this condition being determined post-hence.

### SUMMARY OF THE INVENTION

The techniques described herein provide surgical systems and methods for self-detecting kinematic clamp assemblies. In one aspect, there is a self-detecting kinematic clamp assembly. The self-detecting kinematic clamp assembly detects and indicates a degradation in pose. The self-detecting kinematic clamp assembly includes a tracking element for tracking a pose of the kinematic clamp assembly, a base portion comprising a fixation device that is attachable to a rigid body, a top portion connected to the tracking element, the top portion being removably connected to the base portion, and a detection mechanism. The detection mechanism includes three or more contact points between the base potion and top portion, and a mechanism that detects movement between one or more of the three or more contact points.

In one aspect, there is a computer implemented method. The computer implemented method is for automatically detecting registration degradation. The computer implemented method includes monitoring, by a self-detecting kinematic clamp assembly positioned on a rigid body, a first pose of the self-detecting kinematic clamp assembly for movement between contact points between two portions of the self-detecting kinematic clamp assembly. The computer implemented method includes detecting a change of the first pose of the self-detecting kinematic clamp assembly based on movement of the contact points. The computer implemented method includes transmitting an indication of the change to a computer system wherein the computer system only needs to monitor the self-detecting kinematic clamp assembly to detect the indication.

In one aspect, there is a computer implemented method. The computer implemented method is for automatically detecting registration degradation. The computer implemented method includes detecting data representative of a pose of a self-detecting kinematic clamp assembly positioned on a rigid body. The computer implemented method includes registering the rigid body with a computer generated rigid body model representative of the rigid body based on the detected data. The computer implemented method includes automatically detecting an indication, only from the self-detecting kinematic clamp assembly, representative of a change in the pose of the self-detecting kinematic clamp assembly in relation to the rigid body, wherein the change degrades the registration of the rigid body with the computer generated rigid body model.

In other examples, any of the aspects above can include one or more of the following features. The tracking element can include at least one of a marker and an array of markers. The kinematic clamp assembly can include six contact points arranged so that the detection mechanism can monitor six degrees of freedom of the kinematic clamp assembly. The top portion can be adjustably connected to the tracking element. The kinematic clamp assembly can include a clamp that spherically connects the top portion to the tracking element to allow motion of the tracking element in any axis.

In some examples, the clamp can includes a central member, a first clamp arm hingedly connected to the central member so that the central member and the first clamp arm define a first adjustable spherical receptor, and a tightening mechanism that extends through the first clamp arm and the central member to tighten the first adjustable spherical receptor. The central member can include a first end, a central area, and a second end, and the first clamp arm can be disposed along a top surface of the central member, wherein a first end of the first clamp arm is hingedly connected to the first end of the central member, a second end of the first clamp arm is disposed at the central area of the central member, the top surface of the central member comprising a portion defining a first side of the first adjustable spherical receptor and a bottom surface of the first clamp arm comprising a portion defining a second side of the first adjustable spherical receptor. The clamp can further include a second clamp arm disposed along a bottom surface of the central member, wherein a first end of the second clamp arm is hingedly connected to the second end of the central member, a second end of the second clamp arm is disposed at the central area of the central member, and the bottom surface of the central member and a top surface of the first clamp arm define a second adjustable spherical receptor. The tightening mechanism can extend through the second end of the second clamp arm and the central area of the central member, and terminate at the second end of the first clamp arm.

In other examples, the clamp includes a first clamp side hingedly connected to a second clamp side so that the first clamp side and the second clamp side define a first adjustable spherical receptor, and a tightening mechanism that extends through the first clamp side and terminates at the second clamp side to tighten the first adjustable spherical receptor. The first adjustable spherical receptor can include a lip that protrudes into a space defined by the first adjustable spherical receptor, wherein the lip extends about an inner portion of a top edge of the first adjustable spherical receptor. The first clamp side and the second clamp side can define a second adjustable spherical receptor, wherein the first adjustable spherical receptor is disposed on a bottom portion of the clamp and the second adjustable spherical receptor is disposed on a top portion of the clamp.

In some examples, the base portion, the top portion, or both include one or more magnets. The kinematic clamp assembly can further include a flexible connector connected to the base portion and the top portion. The top portion can be removably connected to the base portion using a Maxwell Mount, a Kelvin Mount, a Canoe Ball/Vee Groove Mount, a Three Tooth Coupling, or any combination thereof. The detection mechanism can include a state indicator that indicates a change in pose based on the detection mechanism. The detection mechanism can include a circuit that drives the state indicator to a first state when each of the three or more contact points are in a first position and a second state based on movement of one or more of the three or more contact points. The circuit can drive the state indicator to the second state based on a separation of one or more of the three or more contact points. The three or more contact points can include an electrically conductive material. The circuit can include a latching logical circuit. The first state can be indicated by a logical high and the second state can be indicated by a logical low or the first state can be indicated by a logical low and the second state can be indicated by a logical high. The state indicator can include an optical transmitter for transmitting a signal representative of the first state, the second state, or both. The optical transmitter can include an LED, IR LCD shutter, or any combination thereof.

In other examples, at least one of the three or more contact points includes a detent assembly, the detent assembly including a detent portion, and a receiving portion that receives the detent portion at least two locations. An equilibrium at a first location of the at least two locations can be less stable than an equilibrium at a second location of the at least two locations. The state indicator can indicate a first state at the first location and a second state at the second location. The second location can be located at a location at which a position of the tracking element changes to a detectable degradation. The receiving mechanism rotates about an axis such that a force applied to the receiving mechanism causes the receiving mechanism to rotate against a force of the detent portion which moves the detent portion from the first location to the second location.

In some examples, each of the three or more contact points includes a load sensor for measuring a load value. The kinematic clamp assembly can further include an indicator to indicate a change in the measured load value. The load sensor can include a strain gauge. The kinematic clamp assembly can further include a circuit, the circuit including, for each load sensor, a strain comparator, wherein the circuit drives a state indicator to a first state when each of the strain comparators indicates a preload value within a predetermined maximum preload and a predetermined minimum preload, and the circuit drives the state indicator to a second state if one or more of the strain comparators indicates a preload value not within the predetermined maximum preload and the predetermined minimum preload.

The techniques, which include both surgical systems and methods, described herein can provide one or more of the following advantages. Advantageously, the occurrence of a registration degradation is immediately realized by the surgical system. By immediately detecting a registration degradation, the surgical system can avoid the surgical procedure being improperly carried out on the patient's anatomy.

Other aspects and advantages of the present invention will become apparent from the following detailed description, taken in conjunction with the accompanying drawings, illustrating the principles of the invention by way of example only.

### BRIEF DESCRIPTION OF THE DRAWINGS

The foregoing and other objects, features, and advantages of the present invention, as well as the invention itself, will be more fully understood from the following description of various embodiments, when read together with the accompanying drawings.

FIG. 1 illustrates an exemplary surgical computer system;

FIG. 2 illustrates an exemplary mechanical tracking system;

FIG. 3 illustrates an exemplary embodiment of a self-detecting kinematic clamp assembly;

FIG. 4 illustrates another exemplary embodiment of a self-detecting kinematic clamp assembly;

FIGS. 5A-5B illustrate an exemplary embodiment of a clamp of a self-detecting kinematic clamp assembly;

FIGS. 6A-6B illustrate another exemplary embodiment of a clamp of a self-detecting kinematic clamp assembly;

FIGS. 7A-7C illustrate an exemplary kinematic clamp component of a self-detecting kinematic clamp assembly;

FIG. 8 illustrates an exemplary embodiment of a detection mechanism of a self-detecting kinematic clamp assembly;

FIG. 9 illustrates another exemplary embodiment of a detection mechanism of a self-detecting kinematic clamp assembly;

FIGS. 10A-10C illustrate another exemplary embodiment of a detection mechanism of a self-detecting kinematic clamp assembly;

FIGS. 11A-11E illustrate another exemplary embodiment of a detection mechanism of a self-detecting kinematic clamp assembly;

FIG. 12 illustrates another exemplary embodiment of a detection mechanism of a self-detecting kinematic clamp assembly; and

FIGS. 13A-13B illustrate computer implemented methods for automatically detecting registration degradation.

### DETAILED DESCRIPTION

In general overview, a self-detecting kinematic clamp assembly self-detects a change in pose, for example, because of the clamp assembly being bumped or hit. Upon detecting a change in pose, the self-detecting kinematic clamp assembly detects and indicates this movement. This indication is used to convey the occurrence of a degradation in registration between a tracked object to which the self-detecting kinematic clamp is coupled and a computer-generated model of the tracked object.

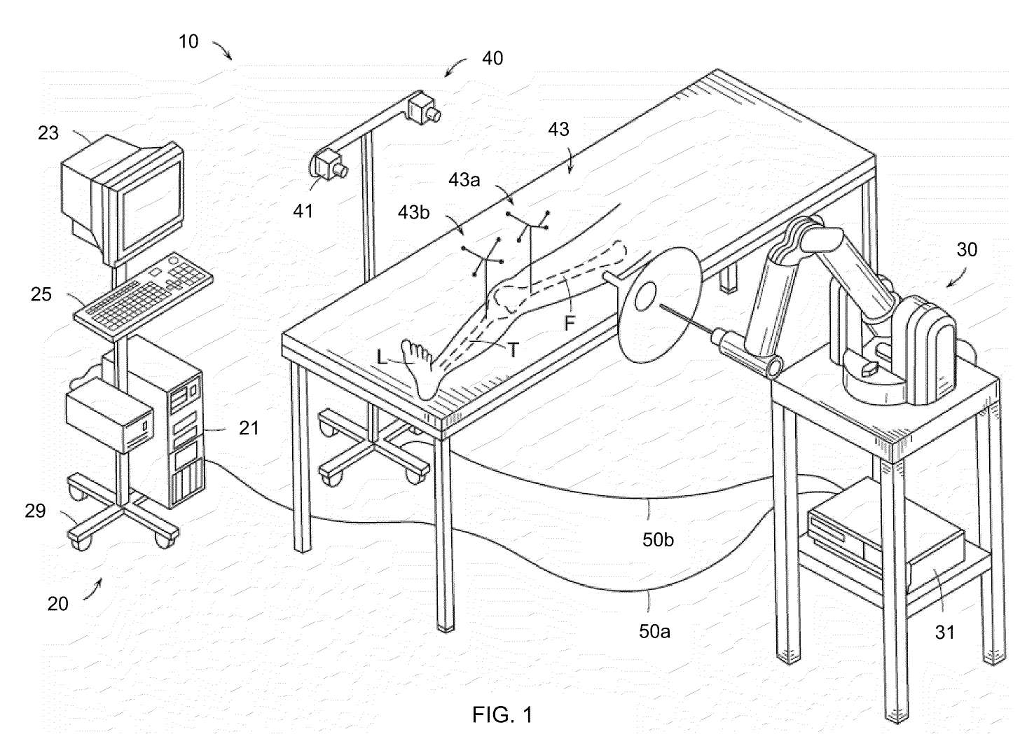

FIG. 1 shows an embodiment of an exemplary surgical computer system 10 in which the self-detecting kinematic clamp assembly and techniques described herein can be implemented. Such an exemplary system is described in detail, for example, in U.S. Patent Application Publication No. 2006/0142657, published Jun. 29, 2006, which is hereby incorporated by reference herein in its entirety. In a preferred embodiment, the surgical computer system is the TACTILE GUIDANCE SYSTEM™ or the RIO™, manufactured by MAKO Surgical Corp., Fort Lauderdale, Fla. The surgical system 10 includes a computing system 20, a haptic device 30, and a tracking (or localizing) system 40. In operation, the surgical system 10 enables comprehensive, preoperative and/or intraoperative surgical planning. The surgical system 10 also provides haptic guidance to a user (e.g., a surgeon) and/or limits the user's manipulation of the haptic device 30 as the user performs a surgical procedure. Although included for completeness in the illustrated embodiment, the haptic device 30 and its associated hardware and software is not necessary to perform the techniques described herein.

The computing system 20 includes hardware and software for operation and control of the surgical system 10. Such hardware and/or software is configured to enable the system 10 to perform the techniques described herein. In FIG. 1, the computing system 20 includes a computer 21, a display device 23, and an input device 25. The computing system 20 may also include a cart 29.

The computer 21 may be any known computing system but is preferably a programmable, processor-based system. For example, the computer 21 may include a microprocessor, a hard drive, random access memory (RAM), read only memory (ROM), input/output (I/O) circuitry, and any other well-known computer component. The computer 21 is preferably adapted for use with various types of storage devices (persistent and removable), such as, for example, a portable drive, magnetic storage (e.g., a floppy disk), solid state storage (e.g., a flash memory card), optical storage (e.g., a compact disc or CD), and/or network/Internet storage. The computer 21 may comprise one or more computers, including, for example, a personal computer (e.g., an IBM-PC compatible computer) or a workstation (e.g., a SUN or Silicon Graphics workstation) operating under a Windows, MS-DOS, UNIX, or other suitable operating system and preferably includes a graphical user interface (GUI).

The display device 23 is a visual interface between the computing system 20 and the user. The display device 23 is connected to the computer 21 and may be any device suitable for displaying text, images, graphics, and/or other visual output. For example, the display device 23 may include a standard display screen (e.g., LCD, CRT, plasma, etc.), a touch screen, a wearable display (e.g., eyewear such as glasses or goggles), a projection display, a head-mounted display, a holographic display, and/or any other visual output device. The display device 23 may be disposed on or near the computer 21 (e.g., on the cart 29 as shown in FIG. 1) or may be remote from the computer 21 (e.g., mounted on a wall of an operating room or other location suitable for viewing by the user). The display device 23 is preferably adjustable so that the user can position/reposition the display device 23 as needed during a surgical procedure. For example, the display device 23 may be disposed on an adjustable arm (not shown) that is connected to the cart 29 or to any other location well-suited for ease of viewing by the user. The display device 23 may be used to display any information useful for a medical procedure, such as, for example, images of anatomy generated from an image data set obtained using conventional imaging techniques, graphical models (e.g., CAD models of implants, instruments, anatomy, etc.), graphical representations of a tracked object (e.g., anatomy, tools, implants, etc.), digital or video images, registration information, calibration information, patient data, user data, measurement data, software menus, selection buttons, status information, and the like.

In addition to the display device 23, the computing system 20 may include an acoustic device (not shown) for providing audible feedback to the user. The acoustic device is connected to the computer 21 and may be any known device for producing sound. For example, the acoustic device may comprise speakers and a sound card, a motherboard with integrated audio support, and/or an external sound controller. In operation, the acoustic device may be adapted to convey information to the user. For example, the computer 21 may be programmed to signal the acoustic device to produce a sound, such as an audible tone or a voice synthesized verbal indication “DONE,” to indicate that a step of a surgical procedure is complete. Similarly, the acoustic device may be used to alert the user to a sensitive condition, such as producing a beep to indicate that a surgical cutting tool is nearing a critical portion of soft tissue.

The input device 25 of the computing system 20 enables the user to communicate with the surgical system 10. The input device 25 is connected to the computer 21 and may include any device enabling a user to provide input to a computer. For example, the input device 25 can be a known input device, such as a keyboard, a mouse, a trackball, a touch screen, a touch pad, voice recognition hardware, dials, switches, buttons, a trackable probe, a foot pedal, a remote control device, a scanner, a camera, a microphone, and/or a joystick.

The computing system 20 is coupled to a computing device 31 of the haptic device 30 via an interface 50a and to a detection device 41 via an interface 50b. The interfaces 50a and 50b can include a physical interface and a software interface. The physical interface may be any known interface such as, for example, a wired interface (e.g., serial, USB, Ethernet, CAN bus, and/or other cable communication interface) and/or a wireless interface (e.g., wireless Ethernet, wireless serial, infrared, and/or other wireless communication system). The software interface may be resident on the computer 21 and/or the computer 31. In some embodiments, computer 21 and 31 are the same computing device.

The system 10 also includes a tracking (or localizing) system 40 that is configured to determine a pose (i.e., position and/or orientation) of one or more objects to detect movement of the object(s). The tracking system 40 can be used, for example, to track movement of anatomy and/or surgical tools during a surgical procedure and/or to track the pose of bones (e.g., a femur F and a tibia T) as a joint is moved through a range of motion. In some embodiments, the tracking system 40 may include a detection device (e.g., the detection device 41) that obtains a pose of an object with respect to a coordinate frame of reference of the detection device. As the object moves in the coordinate frame of reference, the detection device tracks the pose of the object to detect (or enables the surgical system 10 to determine) movement of the object. As a result, the computing system 20 can capture data in response to movement of the tracked object or objects. Tracked objects may include, for example, surgical tools/instruments, patient anatomy, implants/prosthetic devices, and components of the surgical system 10. Using pose data from the tracking system 40, the surgical system 10 is also able to register (or map or associate) coordinates in one space to those in another to achieve spatial alignment or correspondence (e.g., using a coordinate transformation process as is well known). Objects in physical space may be registered to any suitable coordinate system, such as a coordinate system being used by a process running on the computer 21 and/or the computer 31. For example, utilizing pose data from the tracking system 40, the surgical system 10 is able to associate the physical anatomy (i.e., physical space) with a representation of the anatomy (such as an image displayed on the display device 23) (i.e., image space). Based on tracked object and registration data, the surgical system 10 may determine, for example, a spatial relationship between the image of the anatomy and the relevant physical anatomy (i.e., between the image space and the physical space). Knowing this relationship, the image of the anatomy (e.g., displayed on the display device 23) can be made to move in correspondence with the movement of the relevant tracked physical anatomy. The surgical system 10 may also determine, for example, a spatial relationship between the relevant physical anatomy and a surgical tool (not shown) so that the computing system 20 can superimpose (and continually update) a virtual representation of the surgical tool on the image of the anatomy, where the relationship between the virtual representation of the surgical tool and the image of the anatomy is substantially identical to the relationship between the physical surgical tool and the actual physical anatomy. Additionally, by tracking not only the tool but also the relevant anatomy, the surgical system can compensate for movement of the relevant anatomy during the surgical procedure (e.g., by adjusting a virtual object that defines an anatomical cutting boundary in response to the detected movement of the physical anatomy).

Registration may include any known registration technique, such as, for example, image-to-image registration (e.g., monomodal registration where images of the same type or modality, such as fluoroscopic images or MR images, are registered and/or multimodal registration where images of different types or modalities, such as MRI and CT, are registered); image-to-physical space registration (e.g., image-to-patient registration where a digital data set of a patient's anatomy obtained by conventional imaging techniques is registered with the patient's actual anatomy); and/or combined image-to-image and image-to-physical-space registration (e.g., registration of preoperative CT and MRI images to an intraoperative scene). The computer system 10 may also include a coordinate transform process for mapping (or transforming) coordinates in one space to those in another to achieve spatial alignment or correspondence. For example, the surgical system 10 may use the coordinate transform process to map positions of tracked objects (e.g., patient anatomy, etc.) into a coordinate system used by a process running on the computer 31 and/or the computer 21. As is well known, the coordinate transform process may include any suitable transformation technique, such as, for example, rigid-body transformation, non-rigid transformation, affine transformation, and the like.

The tracking system 40 may be any tracking system that enables the surgical system 10 to continually determine (or track) a pose of the relevant anatomy of the patient. For example, the tracking system 40 may comprise a non-mechanical tracking system, a mechanical tracking system, or any combination of non-mechanical and mechanical tracking systems suitable for use in a surgical environment. The non-mechanical tracking system may include an optical (or visual), magnetic, radio, or acoustic tracking system. Such systems typically include a detection device adapted to locate in predefined coordinate space specially recognizable trackable elements (e.g., active or passive markers) that are detectable by the detection device and that are either configured to be attached to the object to be tracked or are an inherent part of the object to be tracked. For example, a trackable element may include an array of markers (also referred to as a tracking array or tracker) having a unique geometric arrangement and a known geometric relationship to the tracked object when the trackable element is attached to the tracked object. The known geometric relationship may be, for example, a predefined geometric relationship between the array of markers and an endpoint and axis of the tracked object. Thus, the detection device can recognize a particular tracked object, at least in part, from the geometry of the markers (if unique), an orientation of the axis, and a location of the endpoint within a frame of reference deduced from positions of the markers. The markers may include any known marker, such as, for example, extrinsic markers and/or intrinsic features of the tracked object. Extrinsic markers are artificial objects that are attached to the patient (e.g., markers affixed to skin, markers implanted in bone, stereotactic frames, etc.) and are designed to be visible to and accurately detectable by the detection device. Intrinsic features are salient and accurately locatable portions of the tracked object that are sufficiently defined and identifiable to function as recognizable markers (e.g., landmarks, outlines of anatomical structure, shapes, colors, or any other sufficiently recognizable visual indicator). The markers may be located using any suitable detection method, such as, for example, optical, electromagnetic, radio, or acoustic methods as are well known. For example, an optical tracking system having a stationary stereo camera pair sensitive to infrared radiation may be used to track markers that emit infrared radiation either actively (such as a light emitting diode or LED) or passively (such as a spherical marker with a surface that reflects infrared radiation). Similarly, a magnetic tracking system may include a stationary field generator that emits a spatially varying magnetic field sensed by small coils integrated into the tracked object.

In some embodiments, as shown in FIG. 1, the tracking system 40 includes a non-mechanical tracking system. In this embodiment, the non-mechanical tracking system is an optical tracking system that comprises a detection device 41 and at least one trackable element (e.g., tracking arrays 43a and 43b) configured to be disposed on (or incorporated into) a tracked object and detected by the detection device 41. In FIG. 1, the detection device 41 includes, for example, a stereo camera pair sensitive to infrared radiation and positionable in an operating room where the surgical procedure will be performed. The tracking arrays 43a and 43b are configured to be affixed to the tracked objects (i.e., the femur F and the tibia T, respectively) in a secure and stable manner and each include an array of markers (e.g., an array S1 in FIG. 15) having a known geometric relationship to the tracked object. The markers may be active (e.g., light emitting diodes or LEDs) or passive (e.g., reflective spheres, a checkerboard pattern, etc.) and preferably have a unique geometry (e.g., a unique geometric arrangement of the markers) or, in the case of active, wired markers, a unique firing pattern. In operation, the detection device 41 detects positions of the markers, and the unique geometry (or firing pattern) and known geometric relationship to the tracked object enable the surgical system 10 to calculate a pose of the tracked object based on the poses of the markers.

The non-mechanical tracking system may include a trackable element for each object the user desires to track. For example, as noted above, in some embodiments, the non-mechanical tracking system includes the anatomy trackers 43a and 43b, generally 43 (to track patient anatomy).

In FIG. 1, the anatomy tracker 43 is disposed on a relevant portion of a patient's anatomy (such as a bone) and is adapted to enable the relevant anatomy to be detected and tracked by the detection device 41. The anatomy tracker 43 includes a fixation device for attachment to the anatomy. The fixation device may be, for example, a bone pin, surgical staple, screw, clamp, wearable device, intramedullary rod, or the like. In some embodiments, the anatomy tracker 43 is configured for use during knee replacement surgery to track the femur F and the tibia T of the patient. In this embodiment, as shown in FIG. 1, the anatomy tracker 43 includes a first tracker 43a adapted to be disposed on the femur F and a second tracker 43b adapted to be disposed on the tibia T. When installed on the patient, the first and second trackers 43a and 43b enable the detection device 41 to detect a pose and track motion of the femur F and the tibia T. As a result, the surgical system 10 is able to detect and capture bone motion in real-time as an individual moves his or her joint through its range of motion.

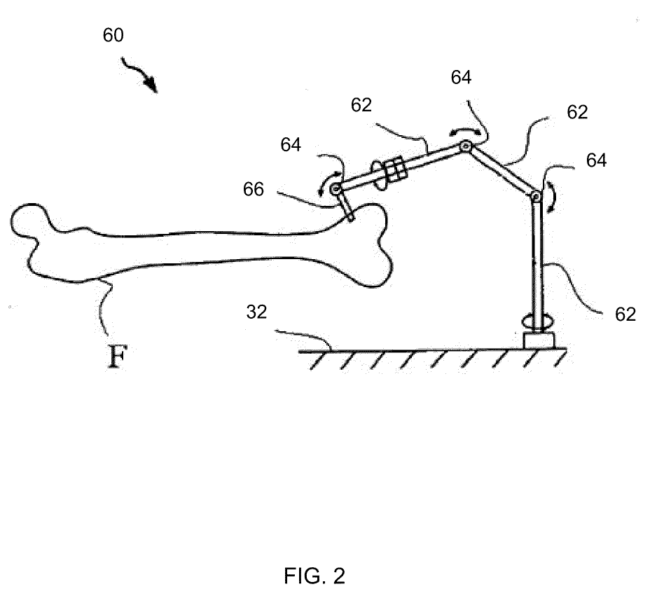

The tracking system 40 may additionally or alternatively include a mechanical tracking system. In contrast to the non-mechanical tracking system (which includes detection device 41 that is remote from the trackers 43), a mechanical tracking system may be configured to include a detection device (e.g., an articulating arm having joint encoders) that is mechanically linked (i.e., physically connected) to the tracked object. The tracking system 40 may include any known mechanical tracking system, such as, for example, a fiber optic tracking system or an articulated arm mechanical tracking system. Examples of a mechanical tracking system are described in U.S. Pat. No. 6,033,415 and U.S. Pat. No. 6,322,567, each of which is hereby incorporated by reference herein in its entirety. In one embodiment, the tracking system 40 includes a mechanical tracking system having a jointed mechanical arm 60 (e.g., an articulated arm having six or more degrees of freedom) adapted to track a bone of the patient. As shown in FIG. 2, the arm 60 has a proximal end affixed to a base 32 of the haptic device 30 and a freely moveable distal end fixed to the femur F of the patient. Alternatively, the proximal end may be affixed to any other suitable location (such as, for example, to a rail of an operating table, a leg holder, etc.) but is preferably connected (e.g., directly or via a bracket) to the base 32 of the haptic device 30 so that the arm 60 moves globally with the haptic device 30. The distal end of the arm 60 includes a fixation device 66 adapted for rigid fixation to the femur F, such as, for example, a bone pin, bone screw, clamp, wearable device, surgical staple, or the like. The arm 60 is configured to have multiple degrees of freedom. For example, in one embodiment, as shown in FIG. 2, the arm 60 includes a plurality of links 62 connected at joints 64. Each joint 64 incorporates one or more position sensors (not shown) to track a pose of the arm 60. The position sensors may include any suitable sensor. In operation, as the femur F moves, the distal end of the arm travels with the femur F. The position sensors (and appropriate software) produce measurements of a pose of the distal end of the arm relative to the proximal end of the arm fixed to the haptic device 30. In this manner, motion of the femur F relative to the haptic device 30 is captured. The mechanical tracking system may also include a second arm that is identical to the arm 60 but is rigidly affixed to the tibia T to enable the tracking system 40 to track motion of the tibia T. In this manner, the mechanical tracking system may be used to track the femur F and the tibia T so that the surgical system 10 can detect bone motion in real time during surgery. Using bone motion data in conjunction with appropriate software, the surgical system 10 can compensate for the bone motion in real time during surgery.

One advantage of a mechanical tracking system over a non-mechanical tracking system is that the detection device (e.g., the arm 60) is physically connected to the tracked object and therefore does not require a line of site to “see” markers on the tracked object. Thus, the user and other personnel may freely move about the operating room during a surgical procedure without worrying about blocking an invisible line of sight between a set of markers and an optical camera. Another advantage of the mechanical tracking system is that the arm 60 may be physically connected to the haptic device 30 (e.g., to the base 32). Such a configuration eliminates the need to track a global or gross position of the haptic device 30 relative to the patient. There is no need to track the global or gross position of the haptic device 30 because the arm 60 moves with the haptic device 30. As a result, the haptic device 30 may be repositioned during a procedure without having to be recalibrated to a bone motion tracking system. Additionally, mechanical tracking systems may be more accurate than non-mechanical tracking systems and may enable faster update rates to the computer 21 and/or the computer 31. Faster update rates are possible because a mechanical tracking system is hardwired to the computer 21 and/or the computer 31. Thus, the update rate is limited only by the speed of the computer 21 and/or the computer 31.

In an alternative embodiment, the arm 60 of the mechanical tracking system may be attached to an operating table, a leg holder, or other structure in the surgical environment. In this embodiment, a calibration is performed to determine a pose of the arm 60 relative to the haptic device 30. For example, in one embodiment, the calibration is performed by placing the distal end of haptic device 30 in a known geometric relationship with the distal end of the arm 60. In another embodiment, the distal end of the arm 60 is placed in a known geometric relationship with the base 32 of the haptic device 30. In yet another embodiment, the distal end of the haptic device 30 is brought into a known geometric relationship with a base of the arm 60.

When the tracking system 40 includes the mechanical tracking system, the arm 60 may be used to register the patient's anatomy. For example, the user may use the arm 60 to register the tibia T while a second arm (e.g., an arm that is identical to the arm 60 but that is affixed to the tibia T) tracks motion of the tibia T. Registration may be accomplished, for example, by pointing a tip of the distal end of the arm 60 to anatomical landmarks on the tibia T and/or by touching points on (or “painting”) a surface of the tibia T with the tip of the distal end of the arm 60. As the user touches landmarks on the tibia T and/or paints a surface of the tibia T, the surgical system 10 acquires data from the position sensors in the arm 60 and determines a pose of the tip of the arm 60. Simultaneously, the second arm provides data regarding motion of the tibia T so that the surgical system 10 can account for bone motion during registration. Based on the bone motion data and knowledge of the position of the tip of the arm 60, the surgical system 10 is able to register the tibia T to the diagnostic images and/or the anatomical model of the patient's anatomy in the computing system 20. In a similar manner, the second arm may be used to register the femur F while the arm 60 (which is affixed to the femur F) tracks motion of the femur F. The patient's anatomy may also be registered, for example, using a non-mechanical tracking system in combination with a tracked probe and/or using the haptic device 30.

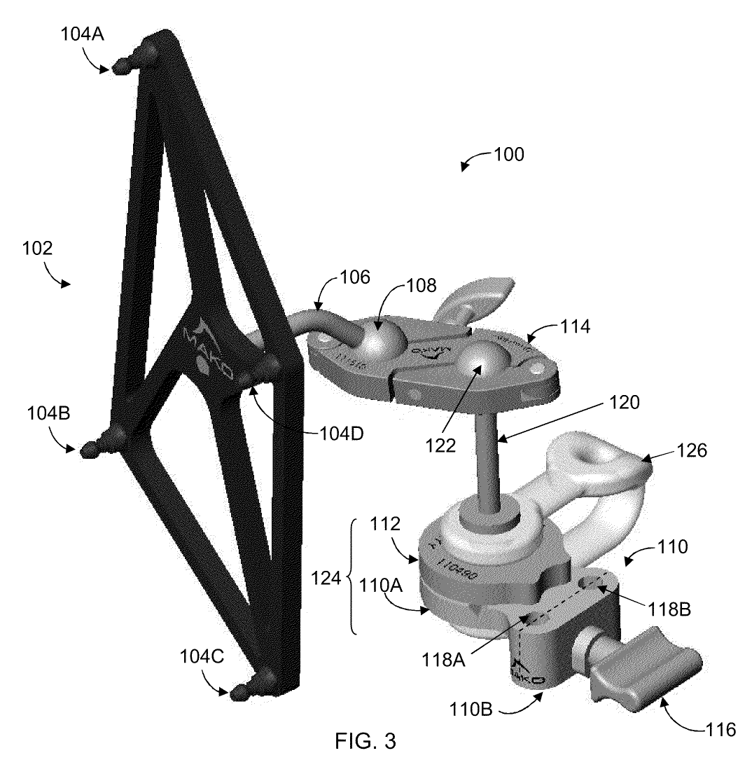

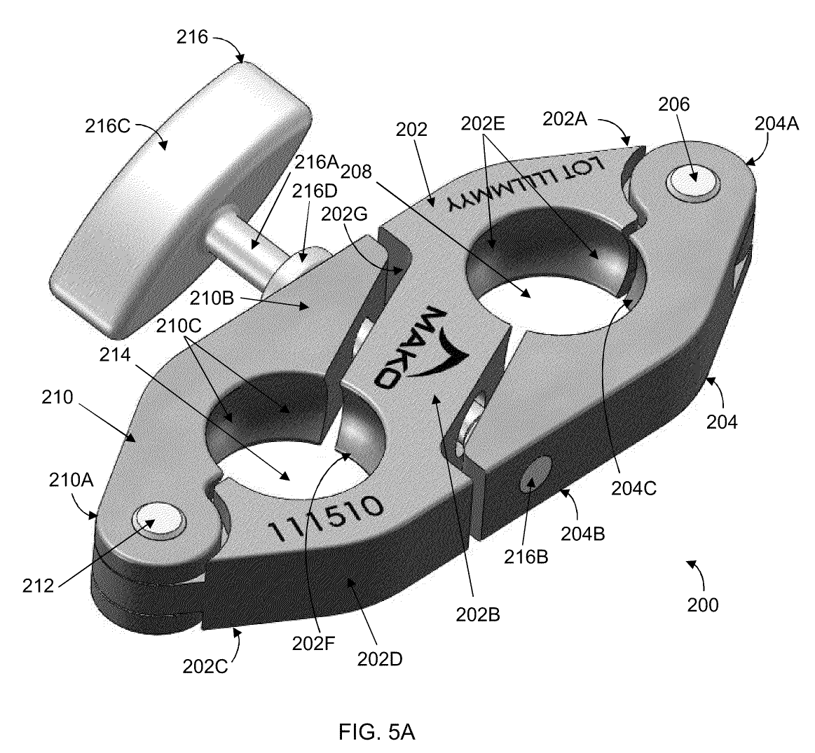

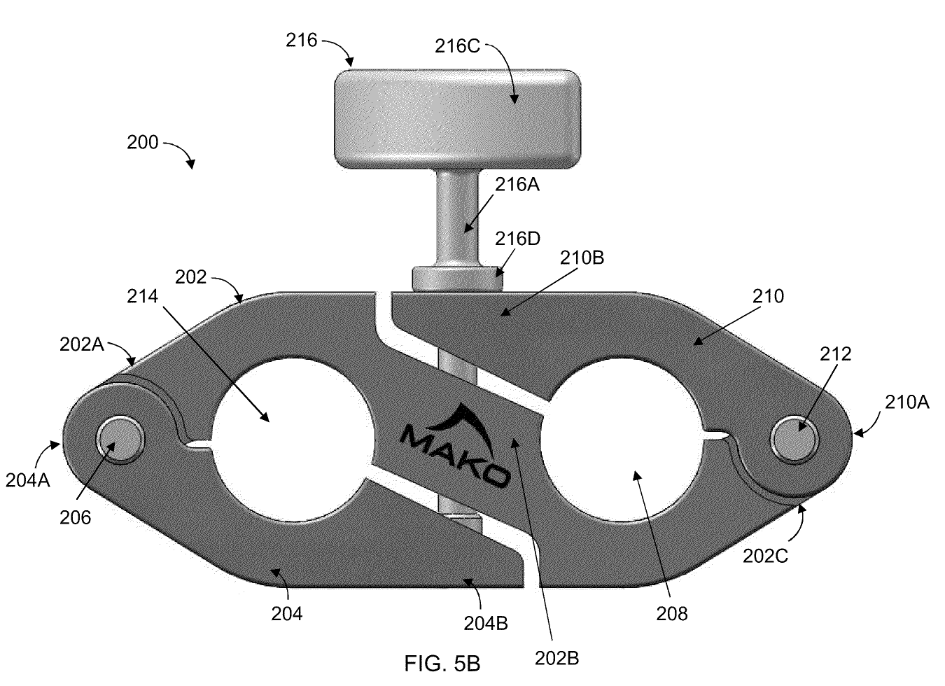

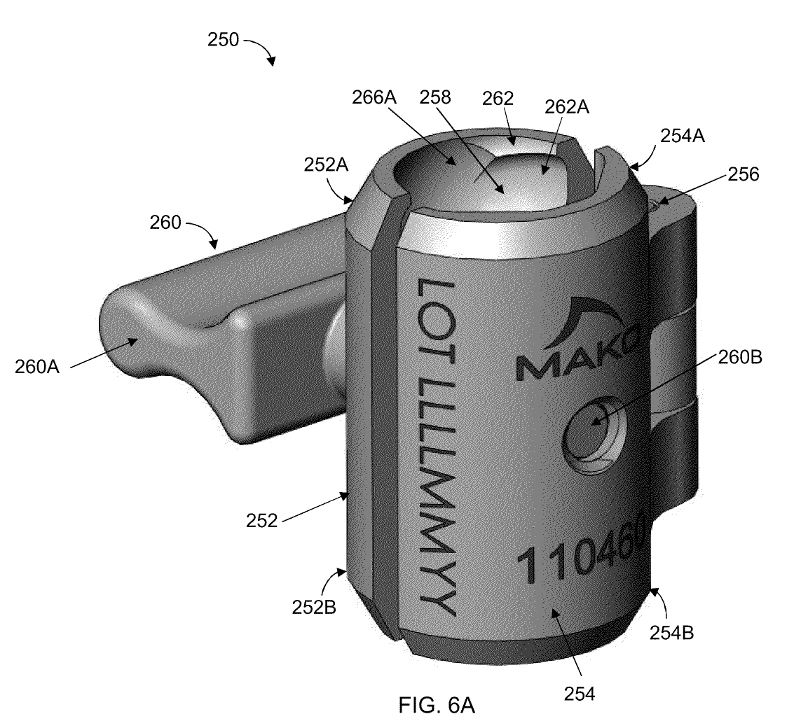

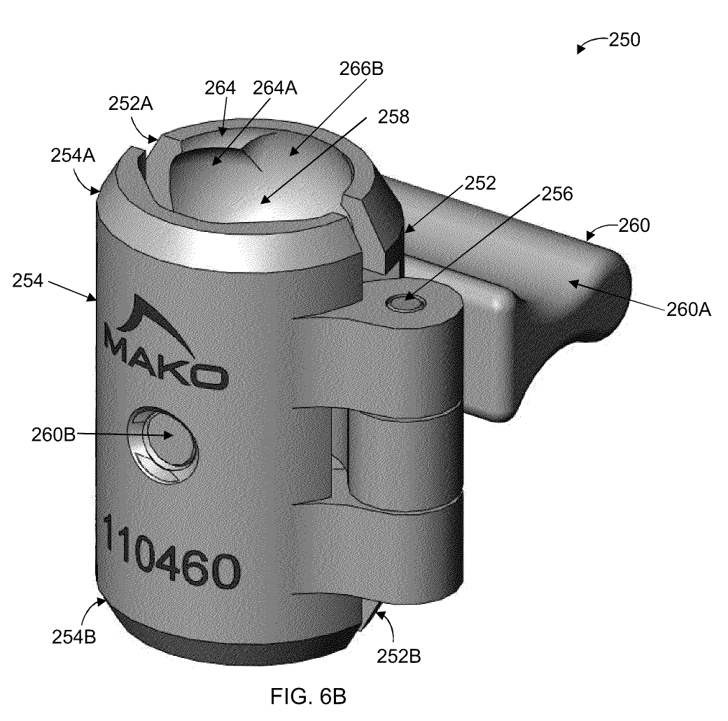

FIG. 3 illustrates an embodiment of a self-detecting kinematic clamp assembly 100 for detecting and indicating a change in pose of a tracking array (or tracking element) relative to a base on which it is mounted, even if the change in pose is only temporary and/or transitory. The self-detecting kinematic clamp assembly 100 includes a tracker 102 (or tracking array or tracking element) for tracking a pose of the kinematic clamp assembly 100. As explained above in connection with the non-mechanical tracking system, the tracker 102 can be an array of markers (not shown) that are detectable by a detection system (e.g., the detection device 41 of FIG. 1) and that have a unique geometric arrangement and a known geometric relationship to a tracked object when the tracker 102 is attached to the tracked object. The known geometric relationship to the tracked object is obtained via the above-described registration process. As long as the geometric relationship between the tracker 102 and the tracked object remains constant, registration is preserved. If the geometric relationship changes, however, registration degrades and must be repeated. As described above, the markers of the tracker 102 may be, for example, passive spheres, retroflective markers, light emitting diodes (LEDs), or the like. The markers may be integral with the tracker 102. Alternatively, the tracker 102 may comprise one or more marker attachment points 104A, 104B, 104C and 104D, collectively marker attachment points 104, adapted to removably receive the markers. The marker attachment points 104 can include, for example, snap-on or threaded fasteners that engage corresponding fastening elements on the markers. In some examples, the tracker 102 is a six degree of freedom (DOF) tracker with four passive reflective spheres from Northern Digital, Inc. The tracker 102 includes a tracker arm 106 extending from the tracker 102 and terminating in a spherical connector 108. As will be explained with reference to FIGS. 5A-6B, the spherical connector allows a high degree of variable positioning of the tracker 102.

In this embodiment, the self-detecting kinematic clamp assembly 100 includes a base portion 110, a top portion 112, and a clamp 114. The base portion 110 includes a fixation device (not shown) that is attachable to a rigid body. For example, the fixation device can be one or more bone pins, surgical staples, screws, or clamps, a wearable device (e.g., a strap), intramedullary rod, or the like. In this embodiment, the base portion 110 is adapted to be removably secured to two bone pins. For example, the base portion 110 may include a first portion 110A, a second portion 110B, and a screw 116. To connect the base portion 110 to a rigid body, the user screws the bone pins into the rigid body, slides the base portion 110 over the bone pins (not shown) so the bone pins extend at least partially into holes 118A, 118B. The user tightens screw 116 to draw the first and second portions 110A and 110B together to thereby securely fix the base portion 110 to the bone pins.

An arm 120 extends from the top portion 112, terminating in a spherical connector 122. The clamp 114 is adapted to spherically connect the top portion 112 to the tracker 102 to allow motion (adjustment) of the tracker 102 in any axis with respect to the top portion 112 by rotating about the spherical connector 122. The clamp 114 is removably secured to the spherical connector 108 of the tracker 102 and the spherical connector 122 of the top portion 112. The clamp 114 is described in further detail with respect to FIGS. 5A-5B.

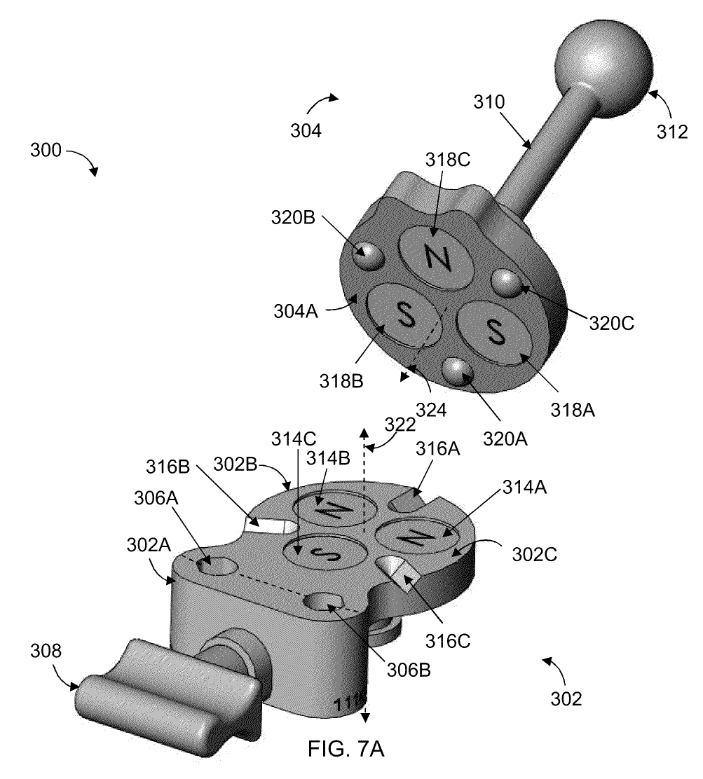

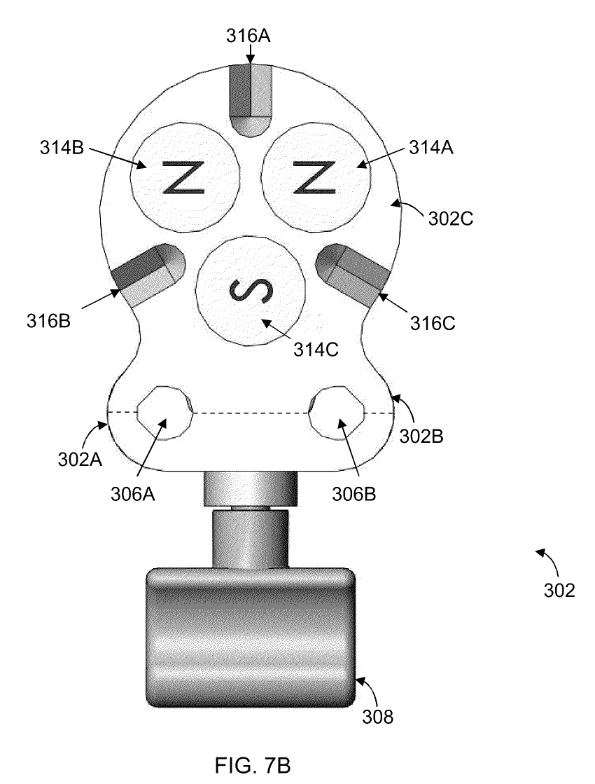

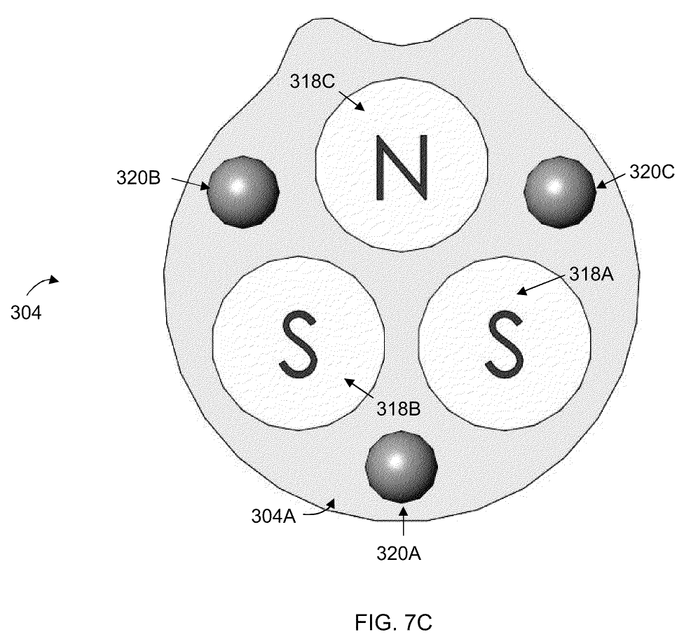

The top portion 112 is removably connected to the base portion 110. The base portion 110 and the top portion 112 comprise a kinematic clamp component 124. The kinematic clamp component 124 can, for example, be configured to allow the tracker 102 to be un-mounted and re-mounted multiple times quickly and with high precision. The kinematic clamp component is described in further detail with reference to FIGS. 7A-7C.

In some embodiments, an optional flexible connector 126 is attached to the base portion 110 and the top portion 112, thereby flexibly coupling the base portion 110 to the top portion 112 without interfering with the connection between the base portion 110 and the top portion 112. In some examples the flexible connector 126 can be configured so that the flexible connector only provides resistance when the base portion 110 and the top portion 112 are separated by a predetermined distance. The flexible connector 126 can be, for example, a silicone or a rubber connector used to flexibly connect the base portion 110 to the top portion 112 so that if the top portion 112 is accidentally disconnected from the base portion 110, the flexible connector 126 limits how far the top portion 112 can fall from the base portion 110 (e.g., prevents the top portion 112 from falling to the floor).

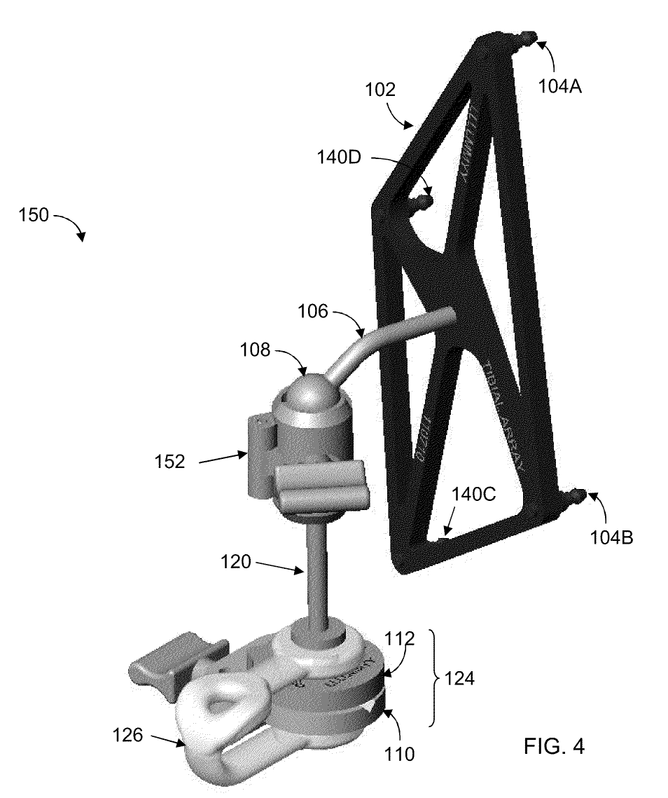

FIG. 4 illustrates another exemplary embodiment of a self-detecting kinematic clamp assembly 150. FIG. 4 illustrates many components of the self-detecting kinematic clamp assembly 100 of FIG. 3 from a different perspective (such components are indicated using the same numbering from FIG. 3). The self-detecting kinematic clamp 150 includes a clamp 152. The clamp 152, like the clamp 114 of FIG. 3, is adapted to spherically connect the top portion 112 to the tracker 102 to allow motion (adjustment) of the tracker 102 in any axis with respect to the top portion 112. The clamp 152 is removably secured to the spherical connector 108 of the tracker 102 and the spherical connector 122 (not visible in the FIG. 4 view) of the top portion 112. The clamp 152 is described in further detail with respect to FIGS. 6A-6B.

While particular reference is made to certain embodiments of the clamps 114 and 152 below in FIGS. 5A-6B, one skilled in the art can appreciate that the size and/or the shape of the clamp 114, 152 can be changed without departing from the spirit of the invention. Further, while the self-detecting kinematic clamp assemblies 100, 150 are shown with only one tracker 102, the kinematic clamp assemblies 100, 150 can be adapted to use any number of trackers. In some embodiments the kinematic clamp component 124 and the clamp 114 or 152 are used separately (e.g., the arm 120 extends from the top portion 112 of the kinematic clamp component 124 and terminates directly with tracker 102, or the kinematic clamp component 124 is omitted such that the arm 120 terminates with the spherical connector 122 on a first end for connection with the clamp assembly 100 or 150 and with a fixation device, such as a bone pin, on the second end). In some embodiments, the self-detecting kinematic clamp assemblies 100, 150 can be used with any tracking system (e.g., with either a non-mechanical tracking system and/or a mechanical tracking system). For example, the arm 120 extends from the top portion 112 of the kinematic clamp component 124 and joins to an arm of a mechanical tracker (e.g., via a joint 64).

FIGS. 5A-5B illustrate an exemplary embodiment of a clamp 200 (e.g., the clamp 114 of the self-detecting kinematic clamp assembly 100 of FIG. 3). FIGS. 5A and 5B illustrate different views of the clamp 200 and thus the following description is generally applicable and refers to either view. The clamp 200 includes a central member 202. The central member 202 includes a first end 202A, a central area 202B, and a second end 202C. A first clamp arm 204 is disposed along a top surface 202D of the central member 202. A first end 204A of the first clamp arm 204 is hingedly connected to the first end 202A of the central member 202 by a pin 206 about which the ends 202A and 204A rotate, thus creating a hinged assembly. A second end 204B of the first clamp arm 204 is disposed at the central area 202B of the central member 202. The central member 202 and the first clamp arm 204 define a first adjustable spherical receptor 208. The top surface 202D of the central member 202 includes a portion 202E defining a first side of the first adjustable spherical receptor 208, and a bottom surface of the first clamp arm 204 includes a portion 204C defining a second side of the first adjustable spherical receptor 208.

A second clamp arm 210 is disposed along a bottom surface 202G of the central member 202. A first end 210A of the second clamp arm 210 is hingedly connected to the second end 202C of the central member 202 by a pin 212 about which the ends 210A and 202C rotate. A second end 210B of the second clamp arm 210 is disposed at the central area 202B of the central member 202. A portion 202F of the bottom surface of the central member 202 and a portion 210C of the top surface of the first clamp arm 210 define a second adjustable spherical receptor 214. A tightening mechanism 216 comprises a shaft 216A that extends through the second end 210B of the second clamp arm 210 and the central area 202B of the central member 202, and a distal end 216B of the shaft 216 terminates at the second end 204B of the first clamp arm 204. The tightening mechanism 216 includes a handle 216C and a stopper portion 216D so that when the handle 216C is turned (by a user), threads disposed about the shaft 216A (not visible in the views of FIGS. 5A or 5B) operate to bring the second end 210B of the second clamp arm 210 toward the central portion 202B of the central member 202 and to bring the second end 204B of the first clamp arm 204A toward the central portion 202B of the central member 202, thereby closing (tightening) the first and second adjustable spherical receptors 208, 210.

The first and second adjustable spherical receptors 208, 214 are configured to be removably secured to a spherical connector (e.g., the spherical connector 108, 122 of FIGS. 3 and 4). The surfaces of the central member 202 and the first and second clamp arms 204 and 210 that define the adjustable spherical receptors 208, 214 (e.g., the portion 202E of the top surface 202D of the central member 202 and the portion 204C of the bottom surface of the first clamp arm 204, and the portion 202F of the bottom surface 202G of the central member 202 and the portion 210C of the top surface of the first clamp arm 210, respectively) are concave in shape. The surfaces can be designed to substantially match the external curvature of a spherical connector. To secure a spherical connector within an adjustable spherical receptor (e.g., 208, 214) of the clamp 200, the user inserts the adjustable spherical connector within the adjustable spherical receptor. The user tightens the tightening mechanism 216 to draw the first clamp arm 204, the second clamp arm 210, and the central member 202 together to thereby securely fix the spherical connector within the spherical receptor.

The surfaces of the spherical receptors (e.g., the portion 202E and portion 204C that define the first adjustable spherical receptor 208, and the portion 202F and portion 210C that define the second adjustable spherical receptor 214) are configured to encompass a sufficient external curvature of the spherical connector so that when the spherical connectors are inserted and the clamp 200 is tightened (e.g., via the tightening mechanism 216) the spherical connectors are securely held in place by the spherical receptors. Advantageously, the clamp 200 can be loosened slightly to allow the spherical connector to rotate in any axis without allowing the spherical connector to be separated from the clamp 200. While spherical connectors and spherical receptors are shown in the figures to allow for rotation in any axis, various shapes can be used for the connectors and receptors to allow for movement (e.g., rotation, translation) in one or more axes (e.g., cylindrically-shaped connectors to allow for rotational movement in one axis and translation along another axis). Further, the surfaces that comprise the adjustable spherical receptors need not be substantially curved, but can be any shape sufficient to securely hold the spherical receptors in place (e.g., the surfaces of the spherical receptors can be shaped so that only an upper lip and a lower lip of the surfaces of the spherical receptor contact the spherical connectors, providing two contact areas sufficient to hold the spherical receptor in place when the clamp 200 is tightened about the spherical connector).

FIGS. 6A-6B illustrate another exemplary embodiment of a clamp 250 (e.g., the clamp 152 of the self-detecting kinematic clamp assembly 150 of FIG. 4). FIGS. 6A and 6B illustrate different views of the clamp 250 and thus the following description is generally applicable and refers to either view, unless otherwise stated. The clamp 250 includes a first clamp side 252 hingedly connected to a second clamp side 254 by a pin 256 about which the first clamp side 252 and the second clamp side 254 rotate so that a top portion 252A of the first clamp side 252 and a top portion 254A of the second clamp side 254 define a first adjustable spherical receptor 258. A bottom portion 252B of the first clamp side 252 and a bottom portion 254B of the second clamp side 254 define a second adjustable spherical receptor (not shown). The second adjustable spherical receptor can be designed as described below with reference to the first adjustable spherical receptor 258. A tightening mechanism 260 includes a handle 260A and a shaft (not shown) which extends through the first clamp side 252. A bottom portion 260B of the shaft terminates at the second clamp side 254 so that when the handle 260A is turned (by a user), threads disposed about the bottom portion 260B of the shaft operate to bring the first clamp side 252 and the second clamp side 254 together, thereby closing (tightening) the first adjustable spherical receptor 258 and the second adjustable spherical receptor, although not shown.

As shown in FIG. 6A, the first adjustable spherical receptor 258 (e.g., and the second adjustable spherical receptor, although not shown) includes a lip 262 that protrudes into a space defined by the first adjustable spherical receptor 258. The lip 262 extends about an inner portion of a top edge of the first adjustable spherical receptor 258 (e.g., the lip 262 extends within a portion of the top portion 252A of the first clamp side 252 and/or a portion of the top portion 254A of the second clamp side 254). As shown in FIG. 6B, the first adjustable spherical receptor 258 (e.g., and the second adjustable spherical receptor, although not shown) comprises a second lip 264 that protrudes into a space defined by the first adjustable spherical receptor 258. The lip 264 extends about an inner portion of a top edge of the first adjustable spherical receptor 258 (e.g., the lip 264 extends about a portion of the top portion 252A of the first clamp side 252 and/or a portion of the top portion 254A of the second clamp side 254). While FIGS. 6A and 6B depict a space 266 (a space 266A, 266B) between the first lip 262 and the second lip 264, in some embodiments the first lip 262 and the second lip 264 are not separated by the space 266, thus forming one continuous lip about the top edge of the first adjustable spherical receptor 258. In some embodiments, the lip can be made of any number of discrete lip portions (e.g., with a third lip, fourth lip, etc.).

In some embodiments, an inner portion 262A of the lip 262 (and/or an inner portion 264A of the lip 264) is curvedly shaped (e.g., to substantially or partially match the external curvature of a spherical connector). In some embodiments, the first adjustable spherical receptor 258 includes a lower lip (not shown) to limit how deep a spherical connector is allowed to travel when inserted into the first adjustable spherical receptor. The lower lip can also be designed so that when the first adjustable spherical receptor 258 is tightened around an inserted spherical connector, the inner portion (not shown) of the lower lip and the inner portion 262A of the first lip 262 and the inner portion 264A of the second lip 264 substantially match the curvature of the spherical connector. The lower lip can comprise any number of lip portions as described with reference to lips 262 and 264.

FIGS. 7A-7C illustrate an exemplary kinematic clamp component 300 of the self-detecting clamp assembly (e.g., the kinematic clamp component 124 of the self-detecting kinematic clamp assembly 100, 150 of FIGS. 3 and 4). FIGS. 7A, 7B, and 7C illustrate different views of the clamp component 300 and thus the following description is generally applicable and refers to any of the views. The kinematic clamp component 300 includes a base portion 302 and a top portion 304. As described with reference to FIG. 3, the base portion 302 is adapted to be removably secured to two bone pins (not shown) with a screw 308, where the base 302 includes a first portion 302A and a second portion 302B that include portions which define holes 306A and 306B into and/or through which the bone pins extend. An arm 310 extends from the top portion 304, terminating in a spherical connector 312. The base portion 302 comprises magnets 314A, 314B and 314C, collectively magnets 314, and grooves (e.g., v-grooves) 316A, 316B and 316C, collectively grooves 316. The top portion 304 comprises magnets 318A, 318B and 318C, collectively magnets 318, and balls 320A, 320B and 320C, collectively balls 320. In some embodiments, the balls 320 are steel balls.

The magnets 314 of the base portion 302 and the magnets 318 of the top portion 304 are positioned so that when the top surface 302C of the base portion 302 is brought into close proximity with the bottom surface 304A of the top portion 304, the magnets 318 are oriented over the magnets 314 (e.g., the magnet 314A is substantially aligned with the magnet 318A, the magnet 314B is substantially aligned with the magnet 318B, and the magnet 314C is substantially aligned with the magnet 318C). The magnets 314, 318 are oriented such that opposite poles of the magnets 314, 318 are placed in close proximity (e.g., the north pole (N) of the magnet 314A is placed in close proximity to the south pole (S) of the magnet 318A, the north pole (N) of the magnet 314B is placed in close proximity to the south pole (S) of the magnet 318B, and the south pole (S) of the magnet 314C is placed in close proximity to the north pole (N) of the magnet 318C) so that the base portion 302 becomes kinematically assembled (e.g., magnetically fastened, removably connected) to the top portion 304. The magnets 314 and 318 enable quick assembly and disassembly of the base portion 302 from the top portion 304. As shown in FIGS. 7A-7C, in some embodiments the magnets 318 are oriented over the magnets 314 such that one or more of the magnets 318 would oppose one or more of the magnets 314 if base portion 302 and the top portion 304 are misaligned. For example, if the top portion 304 is rotated such that magnet 318A is oriented over magnet 314C, the south pole (S) of each magnet causes the top portion 304 to oppose the base portion 302. Advantageously, the magnets 318 and 314 can be configured such that each of the magnets 318 is attracted to a corresponding magnet 314 only when the base portion 302 and the top portion 304 are in the proper orientation.

The grooves 316 and the balls 320 are arranged symmemetrically around a central axis 322, 324 of each portion 302, 304. When the top surface 302C of the base portion 302 is brought into close proximity to the bottom surface 304A of the top portion 304, the balls 320 are oriented over the grooves 316 so that the balls 316 sit in the corresponding grooves 316 (e.g., the ball 320A sits in the groove 316A). Advantageously, in some embodiments the alignment of the balls 320 and the grooves 316 ensures that each time the top portion 304 is disconnected, and then re-connected to the base portion 302, the top portion 304 returns to exactly the same position. Thus, a specific assembly configuration of the base portion 302 to the top portion 304 can be easily preserved regardless of the number of times the top portion 304 is connected to or removed from the base portion 302. The balls 320 should be of sufficient size so that when the base portion 302 is kinematically assembled to the top portion 304, there is a slight gap between the top surface 302C of the base portion 302 and the bottom surface 304A of the top portion 304.

FIGS. 7A-7C show three balls 320 and three corresponding grooves 316, which result in a true kinematic mount. This results in a true kinematic mount because the six degrees of freedom of the kinematic clamp component 300 are constrained by six points of contact. However, other configurations can also be used to removably connect the base portion 302 to the top portion 304 without departing from the principles described herein. For example, the grooves 316 can be located on the top portion 304 and the balls 320 can be located on the base portion 302. In some examples, the top portion 304 can be removably connected to the base portion 302 using a Maxwell Mount, a Kelvin Mount, a Canoe Ball/Vee Groove Mount, a Three Tooth Coupling, or any other type of removable connection. In some embodiments, if departing from a true kinematic mount, care should be taken to not over-constrain the design, which can result in increased stresses on the kinematic clamp assembly (e.g., the self-detecting kinematic clamp assembly 100 of FIG. 3) and an increase in the wear and tear on the components (e.g., the grooves 316 of the top portion 304 and the balls 320 of the base portion 302). Additionally, the kinematic clamp component 300 can include any number of magnets to kinematically assemble the base portion 302 to the top portion 304 (e.g., four magnet pairs).

Advantageously, because the kinematic clamp component 300 allows the top portion 304 to be repeatedly and accurately attached to and removed from the base portion 302, trackers can be removed from their bases whenever necessary or appropriate, such as before impaction of other surgical components. For example, the placement of implants in human anatomy (e.g., in bone and/or cartilage) often requires a considerable use of force using hammers and other impaction devices. During such an impaction, the force exerted on an implant can be transferred to the self-detecting kinematic clamps in the vicinity. Removing the relevant trackers prevents the trackers from being affected by the impaction of the surgical components. Additionally, because the top portion 304 can break away from the base portion 302 if subjected to a sufficient force, the base portion 302 (and the connection to the rigid body (e.g., the connection to a bone via the bone pins)) can be protected from a load applied to the top portion 304 (e.g., an inadvertent bump), which preserves the registration.

The kinematic clamp component (e.g., the kinematic clamp component 300 of FIGS. 7A-7C) can include a detection mechanism. The detection mechanism can include a state indicator that indicates a change in pose of the kinematic clamp component based on the detection mechanism. FIG. 8 illustrates an exemplary embodiment of a detection mechanism 350 for a kinematic clamp component of a self-detecting kinematic clamp assembly. The detection mechanism 350 includes a mechanism 352 for detecting and indicating two states of the kinematic clamp component. The mechanism 352 includes a circuit 353, as indicated by reference numerals 353A through 353G. The mechanism 352 includes a latch 354. The latch 354 can instantaneously respond to the pose degradation occurrence and stores information indicative of the pose degradation in the latch 354 until the information is read from the latch 354 by the system (e.g., the computing system 20 of FIG. 1). In some embodiments, if the system is configured to discretely sample the latch 354, the degradation “event” can occur between samples. Subsequent to pose restoration, the latch 354 can be reset to indicate a valid pose/registration condition. The latch 354 has a trigger input 356 and a reset input 358. The trigger input 356 includes three channels, channel (1) 364, channel (2) 366 and channel (3) 368 in series with each other. The system voltage Vₛ is input into the circuit 353 at 353A, which is connected to the latch 354 at 360 (e.g., to provide a voltage source to the latch 354) and is connected to the trigger input 356 through a resistor 362.

Channel (1) 364 includes a first conductor 364A and a second conductor 364B, channel (2) 366 includes a first conductor 366A and a second conductor 366B, and channel (3) 368 includes a first conductor 368A and a second conductor 368B. A ball 370 fits into channel 364 to create two contact points, a contact point 376A between the first conductor 364A and the ball 370 and a contact point 376B between the second conductor 364B and the ball 370. A ball 372 fits into channel 366 to create two contact points, a contact point 378A between the first conductor 366A and the ball 372 and a contact point 378B between the second conductor 366B and the ball 372. A ball 374 fits into channel 368 to create two contact points, a contact point 380A between the first conductor 368A and the ball 374 and a contact point 380B between the second conductor 368B and the ball 374. An output 382 of the latch 354 is connected to an optical transmitter 384. Signals from the optical transmitter 384 are monitored by a monitoring device 386.

The contact points (e.g., for channel (1) 364, the first conductor 364A, the second conductor 364B, and the ball 370) include an electrically conductive material so current can flow through the contact points. The balls 370, 372 and 374 and the conductors 364A, 364B, 366A, 366B, 368A, and 368B can be made from any conductive material, such as steel. Alternatively, the balls 370, 372 and 374 and the conductors 364A, 364B, 366A, 366B, 368A, and 368B can be made from a non-conductive material, but be coated with a conductive coating, or have a surface of conductive material so that contact between the balls 370, 372 and 374 and the conductors 364A, 364B, 366A, 366B, 368A, and 368B complete an electrical circuit and allow current to flow.

The latch 354 can be any kind of bistable multivibrator electronic circuit (e.g., an SR latch, a JK latch, a D latch, a flip flop, a chain of two or more latches, and the like) with two stable states and may include additional logic circuitry (such as AND gates, OR gates, invertors, one or more additional latches, and the like), depending on what circuit or device the output 382 is driving and the configuration of the circuit connected to the trigger input 356. The optical transmitter 384 can be, for example, a light emitting diode (LED), an infrared liquid crystal display (IR LCD) shutter, and/or the like. In some examples, the optical transmitter 384 is omitted and the output 382 of the mechanism 352 is used in other ways to indicate the state of the circuit 353 (e.g., as a logic high or logic low voltage signal, as a sound generating device that indicates a certain state of the output of latch 354 in an auditory way, such as a beep or buzz, or conveyed to a monitoring device 386 via a wire link as in the case of a mechanical tracking and other like indications). In some embodiments, the output 382 is used as a signal to turn off the surgical tool or to perform other evasive actions.

The mechanism 352 can monitor the contact points and detect a break in any of the contact points. For example, with reference to FIGS. 7A-7C, the mechanism 352 can be configured to monitor the contact locations between the balls 320 of the top portion 304 and the grooves 316 of the base portion 302 (as will be explained in detail below). Breaks in the contact locations would be caused by movement of the top portion 304 away from the base portion 302. The mechanism 352 latches the existence of the break in pose integrity (e.g., stores the state indicating the occurrence of a degraded state, and outputting a signal indicating such occurrence until being reset) via the latch 354 to indicate that the movement of the top portion 304 may have degraded the registration between a rigid body tracked object (e.g., a bone) and a computer generated model of the rigid body. The indication from the latch 354 indicates the movement to a surgeon, who knows the kinematic clamp assembly should be checked to verify whether the registration was degraded by the motion. Subsequent to the indication of a degradation, the pose of the kinematic mount must be restored and the latch 354 must be reset to a valid registration state.

In operation, the latch 354 indicates a first state when the circuit 353 is closed and a second state when the circuit 353 is opened. The system voltage Vₛ is input into the circuit 353 at 353A. In some examples, Vₛ is approximately +5V or +3V. The latch 354 is also connected to ground (V_{D}, or 0V). If the circuit 353 is closed (e.g., all the contact points 376A-376B, 378A-378B, and 380A-380B are connected), the voltage along the trigger input 356 is 0V. For example, if the circuit 353 is closed, the circuit 353 is grounded to V_{D} and the output 382 of the latch 354 latches to low because the voltage along the trigger input 356 is 0V (or if the latch 354 is inverted, the output 382 of the latch 354 latches to high), indicating the first state of the circuit 353. When any portion of the circuit 353 is opened (e.g., at channel (1) 364) due to a separation of one or more of the contact points (e.g., the contact points 376A, 376B for channel (1) 364), the trigger input 356 is not grounded to V_{D} and is instead pulled up to approximately Vs through the resistor 362 and the latch 354 latches to high (or if the latch 354 is configured to invert the input, the latch 354 latches to low), indicating the second state of the circuit 353. For example, if any of the balls 370, 372 or 374 are moved away from the channel 364, 366 or 368, one or more of the associated contact points is broken, causing the circuit 353 to open. For example, if the ball 370 is separated from the first conductor 364A, then the contact point 376A is broken, which causes the circuit 353 to open.

In some examples, the balls 370, 372 and 374 are located on the top portion of the kinematic clamp component, and the channels 364, 366 and 368 are located on the base portion of the kinematic clamp component. As an example, with reference to FIG. 7A, the balls 370, 372 and 374 correspond to the balls 320 on the top portion 304, and the channels 364, 366 and 368 correspond to the grooves 316 on the base portion 302. For example, channel (1) 364 can be a groove configured such that one side of the groove corresponds to the first conductor 364A and the other side of the groove corresponds to the second conductor 364B. The balls 320, 322 and 324 can be of sufficient size so that when the base portion 302 is kinematically assembled to the top portion 304, there is a slight gap between the top surface 302C of the base portion 302 and the bottom surface 304A of the top portion 304. Because there is a slight gap, the only physical contact points between the base portion 302 and the top portion 304 are the contact points between the balls 320 and the grooves 316 (e.g., the contact points 376A, 376B, 378A, 378B, 380A and 380B of FIG. 8). Alternatively, the top surface 302C of the base portion 302 and the bottom surface 304A of the top portion 304 can be coated with a non-conductive coating such that the only conductive contacts are between the balls 320 and the grooves 316. When the base portion 302 is kinematically assembled to the top portion 304, the balls 320 are in contact with the grooves 316, which completes the circuit 353 and drives the state indicator (e.g., the optical transmitter 384) to a first state (e.g., on or off). When the top portion 304 is moved in a way to separate one or more of the balls 320 from its corresponding groove 316, the circuit 353 is opened, which drives the state indicator to a second state (e.g., off instead of on or on instead of off).

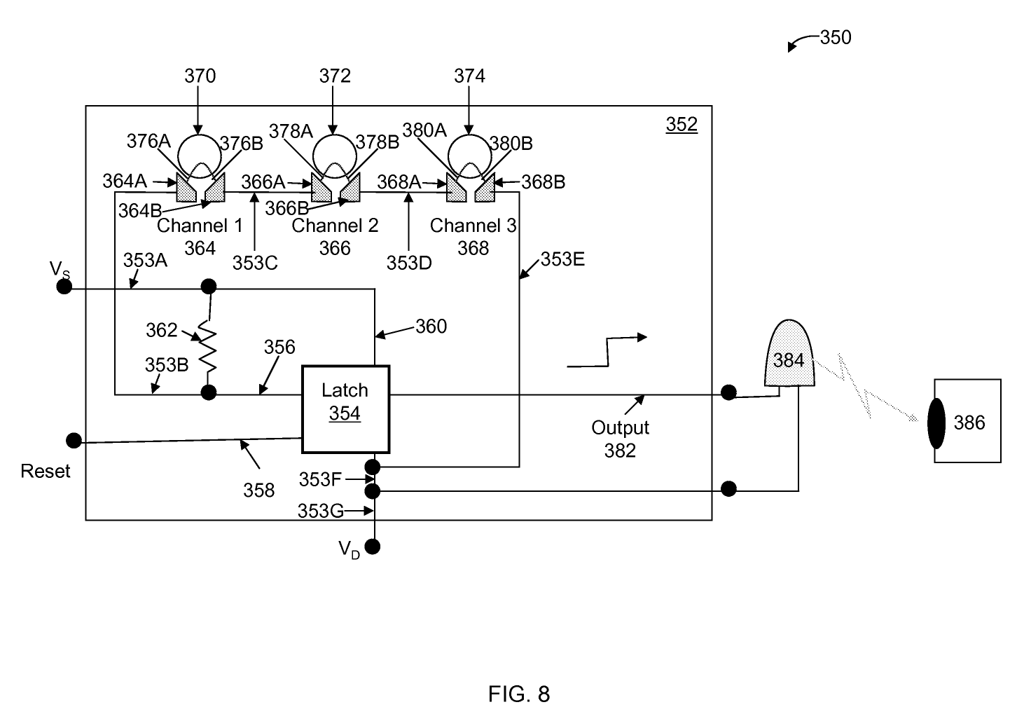

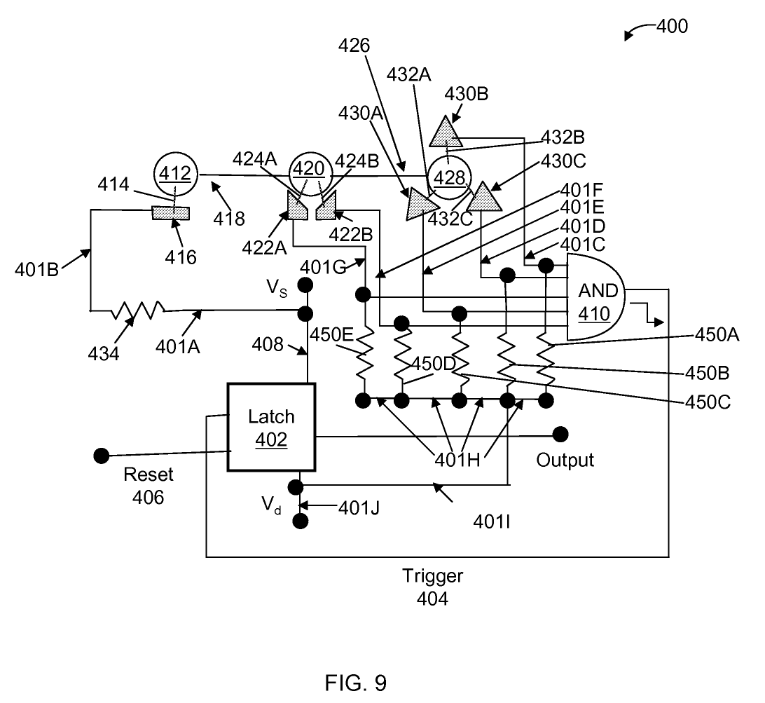

While the channels (channel (1) 364, channel (2) 366, and channel (3) 368) of FIG. 8 are each shown with two conductors, any number of conductors can be used for any number of channels. FIG. 9 illustrates another exemplary embodiment of a detection mechanism 400 of a self-detecting kinematic clamp assembly. The detection mechanism 400 includes a circuit 401 indicated by reference numerals 401A-401J, couplings 418 and 426, and resistors 450A-450E. The detection mechanism 400 includes a latch 402 for indicating the two states of the kinematic clamp component. The latch 402 can instantaneously respond to the pose degradation occurrence and stores information indicative of the pose degradation in the latch 402 until the information is read from the latch 402 by the system. Subsequent to pose restoration, the latch 402 can be reset to indicate a valid pose/registration condition. The latch 402 has a trigger input 404 and a reset input 406. The system voltage Vₛ is input to the latch 402 at 408 and is connected to a resistor 434. The latch 402 is also connected to ground (V_{D}, or 0V). A ball 412 creates a contact point 414 with a conductor 416. The ball 412 is electrically coupled to a ball 420 by the coupling 418. The ball 420 can be in contact with a first conductor 422A and a second conductor 422B to create two contact points, a contact point 424A between the first conductor 422A and the ball 420 and a contact point 424B between the second conductor 422B and the ball 420. The ball 420 is electrically coupled to a ball 428 by the coupling 426. The ball 428 can be in contact with a first conductor 430A, a second conductor 430B, and a third conductor 430C to create three contact points, a contact point 432A between the first conductor 430A and the ball 428, a contact point 432B between the second conductor 430B and the ball 428, and a contact point 432C between the third conductor 430C and the ball 428. This kinematic mount design is sometimes referred to as a Kelvin mount (i.e., cone, vee, flat).

As described above, the contact points include an electrically conductive material so current can flow through the contact points. Each of the inputs to an AND gate 410 are connected to resistors 450A-E, respectively. The other ends of the resistors 450A-E are connected to V_{D}. Each of the inputs to the AND gate 410, and thus one side of each of the resistors 450A-E, is connected to the conductors 430B, 430C, 430A, 422B and 422A, respectively. This configuration creates five parallel circuits, with each corresponding to one of the inputs to the AND gate 410. Each of these parallel circuits is connected through the ball 412, the contact point 414, the conductor 416, and the resistor 434 to the voltage source Vs.

The AND gate 410 is a digital logic gate that indicates a logic HIGH (1) (e.g., voltage Vs, +5V, +3V) only if all the inputs to the AND gate 410 are HIGH (1). If one or more of the inputs to the AND gate 410 are a logic LOW (0) (e.g., voltage V_{D}, 0V), a LOW (0) output results (e.g., to the input trigger 404 of the latch 402). If the circuit 401 is open at any of the contact points, one or more inputs to the AND gate 410 will drop to 0V. For example, if the contact point 424A between the ball 420 and the conductor 422A is opened, the voltage along 401G and thus the input to the AND gate 410 drops to 0V, resulting in a LOW (0) output. The detection mechanism 400 can be configured to monitor the contact points between a kinematic clamp component to detect movement between any of the contact points as described above with reference to FIG. 8. This can advantageously indicate to a surgeon that there could be degradation in the registration due to this movement.

The detection mechanism 400 latches the existence of the break in pose integrity (e.g., stores the state indicating the occurrence of a degraded state, and outputting a signal indicating such occurrence until being reset) via the latch 402. In operation, the illustrated configuration of the detection mechanism 400 drives each input of the AND gate 410 to a logic HIGH when the circuit for each input is closed. For example, if Vₛ is +5V, and there is a very small voltage drop across the resistor 434 (which is an optional resistor) to ˜4.9V at the AND gate input side of the resistors 450A-E. If all the contact points 414, 424A, 424B, 432A, 432B, and 432C created by the balls 412, 420 and 428 are maintained, the current flows such that all inputs to the AND gate 410 are HIGH (1). If the latch 402 receives a HIGH (1) over the trigger 404, the latch 402 indicates a first state (e.g., a logical high). The first state is indicative of the circuit 400 being closed. If one or more of the contact points are open (e.g., by separating the ball 412 from the conductor 416 to break the contact point 414), then current will not flow through at least one or more of the parallel circuits and one or more of the inputs to the AND gate 410 will drop to ground such that one or more inputs to the AND gate are LOW (0). The latch 402 will receive a LOW (0) over the trigger 404, causing the latch 402 to indicate a second state. The second state is indicative of at least one of the contact points 414, 424A, 424B, 432A, 432B, and 432C being open at least for some small amount of time. Thus, the detection mechanism 400 detects movement that causes a break in any of the contact points 414, 424A, 424B, 432A, 432B, and 432C and latches that detection. Subsequent to the indication of a degradation, the pose of the kinematic mount must be restored and the latch 402 must be reset to a valid registration state. The conductors 430B, 430C, 430A, 422B, 422A, and 416 in a Kelvin mount design are configured to deal with force in each of the six degrees of freedom. Thus, use of contact points whose broken connection can be detected in each of these six degrees of freedom ensures that motion in any of the six degrees of freedom is detected, indicating a change in pose, even if only temporary.

It is noted that there are many variations of circuit configurations that can be used to achieve these same results, namely a detection of the break of any of the contact points 414, 424A, 424B, 432A, 432B, and 432C due to some motion of the clamp assembly and a latching of that detection so that the temporary movement can be indicated. For example, different logic devices, different polarities of voltages and logic lows and logic highs can be implemented to achieve these same results. Further, as noted above, the output of the latch 402 can be connected to additional electronics, such as additional electrical circuits, optical devices, or auditory devices so that a change of state can be latched at the output of the latch 402 and indicated to the surgeon in a way that he or she becomes aware of the movement right away.

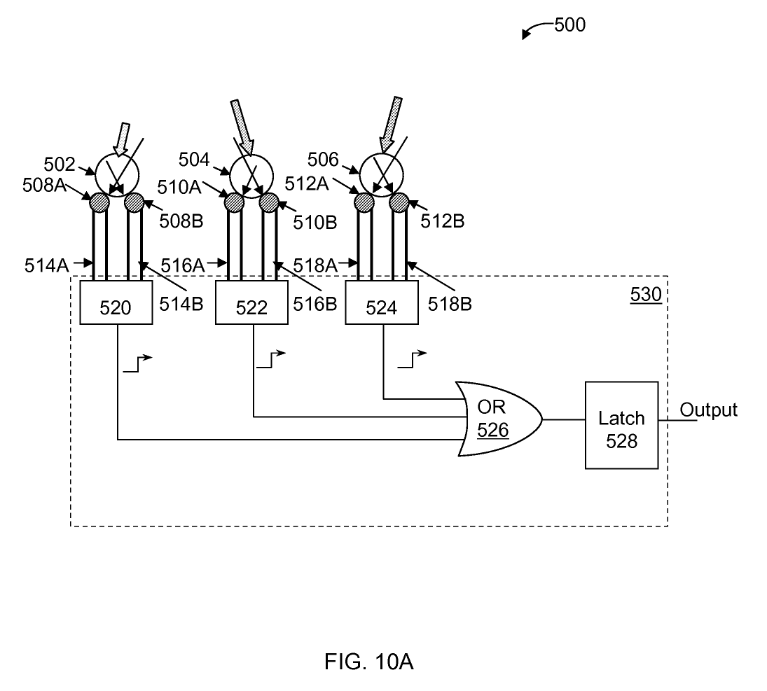

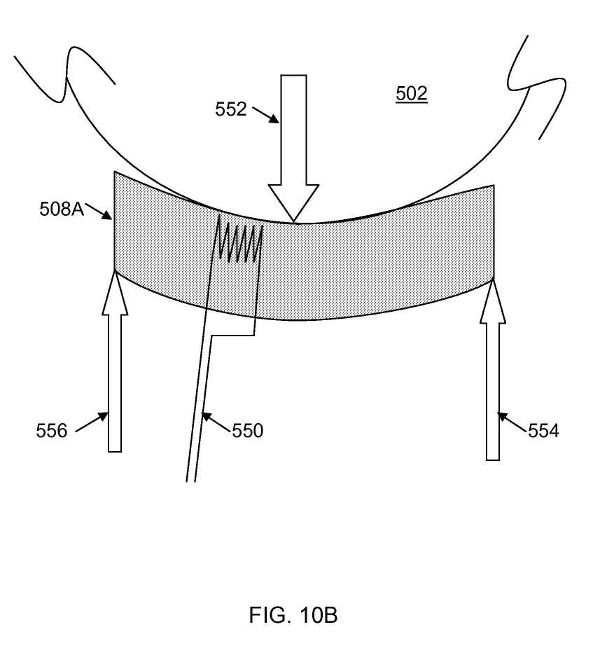

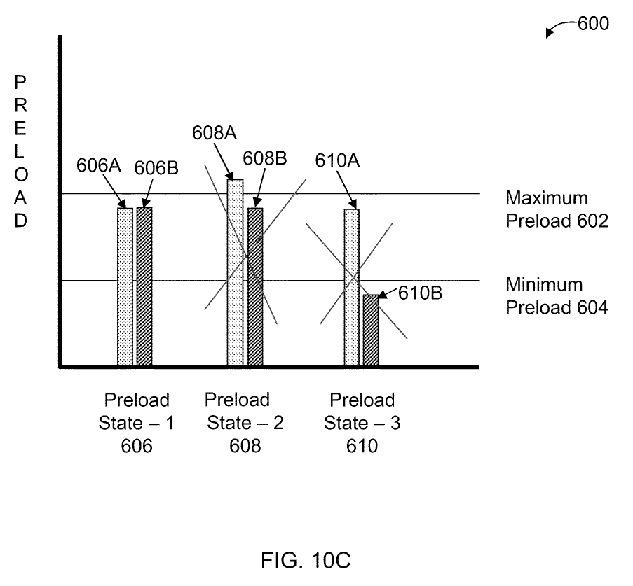

FIGS. 10A-10C illustrate another exemplary embodiment of a detection mechanism 500 of a self-detecting kinematic clamp assembly. The detection mechanism 500 measures a load value from balls 502, 504 and 506. Each ball is in contact with two load sensors for measuring a load value. The ball 502 is in contact with load sensors 508A and 508B, the ball 504 is in contact with load sensors 510A and 510B, and the ball 506 is in contact with load sensors 512A and 512B. Each load sensor includes a strain gauge to measure a load value for the particular load sensor. Each load sensor is supported by a channel (the load sensor 508A is supported by a channel 514A, the load sensor 508B is supported by a channel 514B, the load sensor 510A is supported by a channel 516A, the load sensor 510B is supported by a channel 516B, the load sensor 512A is supported by a channel 518A, and the load sensor 512B is supported by a channel 518B). The channels can be made, for example, of two dowel pins, one pin on each side of the load sensor the channel is supporting. The output of each group of load sensors is compared by a strain comparator, strain comparators 520, 522 and 524. The output of each strain comparator is input to an OR gate 526. The output of the OR gate 526 is input to a latch 528 (e.g., a state indicator). The latch 528 can instantaneously respond to the pose degradation occurrence and stores information indicative of the pose degradation in the latch 528 until the information is read from the latch 528 by the system. Subsequent to pose restoration, the latch 528 can be reset to indicate a valid pose/registration condition. A circuit 530 includes the strain comparators 520, 522 and 524, the OR gate 526, and the latch 528.

FIG. 10B shows a close-up view of the load sensor 508A, which includes a strain gauge 550. The upper load component 552 from the ball 502 causes reaction loads 554 and 556. The upper load component 552 and the reaction loads 554 and 556 cause the load sensor 508A to bend. The strain gauge 550 can measure the load 552 from the ball 502. The strain comparators (e.g., the strain comparator 520, 522 and 524) can be used to monitor the status of a kinematic clamp component. For example, the balls can be located on the top portion of the kinematic clamp component, and the load sensors can be located on the base portion of the kinematic clamp component. The pose of the kinematic clamp component can be a function of the preload (e.g., the anticipated load for a proper pose of the kinematic clamp component) of the top portion on the base portion of the kinematic clamp component. For example, the pose can be affected by a large applied load or when the kinematic clamp component (and/or the kinematic clamp assembly) is subject to acceleration (e.g., movement of the rigid body that the kinematic clamp assembly is connected to). Advantageously, because the pose of a kinematic clamp assembly may be repeatable only within a range of the preload of the top portion on the base portion of the kinematic clamp component, the load sensors can monitor the kinematic clamp component preload.

The circuit 530 latches the existence of the break in pose integrity (e.g., stores the state indicating the occurrence of a degraded state, and outputting a signal indicating such occurrence until being reset) via the latch 528. The preload components are monitored for each contact point (e.g., at the load sensor 508A, 508B, etc.). If the preload is invalid for one or more preload components, the latch 528 (e.g., an indicator) can indicate a change in the measured load value. The circuit 530 drives the latch 528 to a first state when each of the strain comparators 520, 522 and 524 indicates a preload value within a predetermined maximum preload and a predetermined minimum preload. The circuit 530 drives the latch 528 to a second state if one or more of the strain comparators 520, 522 or 524 indicates a preload value that is not within the predetermined maximum preload and the predetermined minimum preload. The OR gate 526 can be any digital logic gate that implements a logical disjunction, where if one or more of the inputs (e.g., from the strain comparators 520, 522 or 524) to the OR gate 526 are HIGH (1), the OR gate 526 outputs a HIGH (1) output. If none of the inputs are HIGH (1), a LOW (0) output results.

For example, with reference to FIG. 10C, a graph 600 illustrates preload measurements for the load sensors of FIG. 10A. The individual values for each load sensor are compared against a predetermined value for a maximum preload 602 and a minimum preload 604. Each group of load sensors for a particular ball comprise a preload state for the particular ball (e.g., the load sensors 508A and 508B comprise preload state (1) 606 for the ball 502, the load sensors 510A and 510B comprise preload state (2) 608 for the ball 504, and the load sensors 512A and 512B comprise preload state (3) 610 for the ball 506). Bar one 606A corresponds to the preload value for the load sensor 508A, bar two 606B corresponds to the preload value for the load sensor 508B, bar three 608A corresponds to the preload value for the load sensor 510A, bar four 608B corresponds to the preload value for the load sensor 510B, bar five 610A corresponds to the preload value for the load sensor 512A, and bar six 610B corresponds to the preload value for the load sensor 512B.

As shown in the graph 600, with respect to preload state (1) 606, the preload values indicated by bars 606A and 606B fall within the maximum preload 602 and the minimum preload 604, so the strain comparator 520 outputs a LOW (0) output to indicate a valid preload. For preload state (2) 608, the preload value indicated by bar 608A is outside of the maximum preload 602, so the strain comparator 522 outputs a HIGH (1) output to indicate an invalid preload even though the preload value indicated by bar 608B is within the maximum preload 602 and the minimum preload 604. Similarly, for preload state (3) 610, the preload value indicated by bar 610B is outside of the minimum preload 604, so the strain comparator 524 outputs a HIGH (1) output to indicate an invalid preload. The OR gate 526 outputs a HIGH (1) output to the latch 528, which can latch low or high depending on how the latch is configured. Subsequent to the indication of a degradation, the pose of the kinematic mount must be restored and the latch 528 must be reset to a valid registration state.

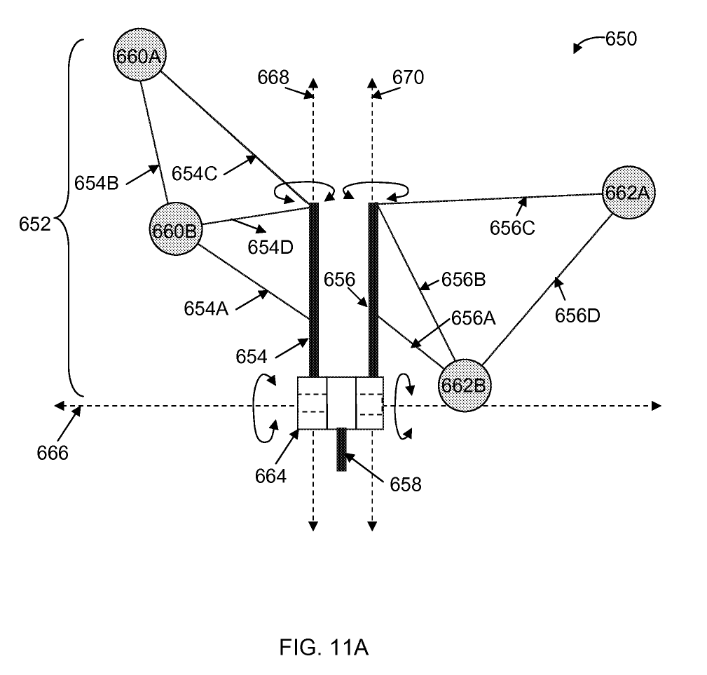

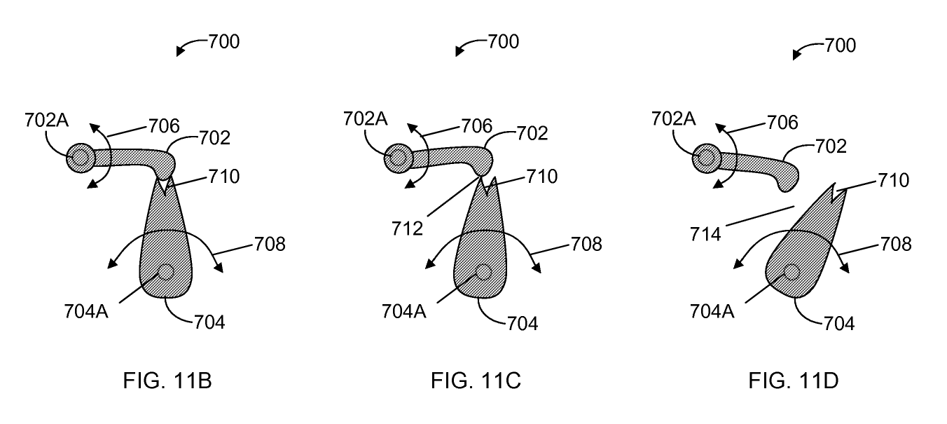

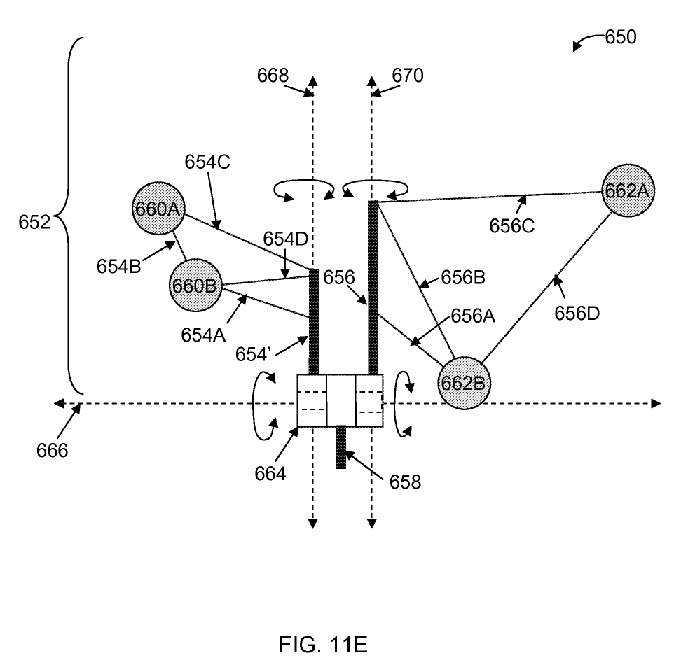

FIGS. 11A-11C illustrate another exemplary embodiment of a detection mechanism 650 of a self-detecting kinematic clamp assembly. The detection mechanism 650 includes a tracker 652. The tracker 652 includes two tracker arms 654 and 656 and a mounting arm 658. The mounting arm 658 can be mounted directly to a rigid body (e.g., a bone) or terminate in a spherical connector for use with a kinematic clamp assembly as described above with reference to FIGS. 3-4. The tracker arm 654 includes supports 654A-654D connected to markers 660A and 660B. The tracker arm 656 includes supports 656A-656D connected to markers 662A and 662B. The detection mechanism 650 includes a detent component 664, comprising detent assemblies (e.g., contact points, not shown) for each tracker arm 654, 656. The tracker arm 654 can rotate about an axis 666 and an axis 668. The tracker arm 656 can rotate about the axis 666 and an axis 670.

FIGS. 11B-D illustrate an exemplary detent assembly 700. The detent assembly 700 includes a detent portion 702 and a receiving portion 704 that receives the detent portion 702 at least two locations. The detent portion 702 is rotatable about a point 702A in a direction indicated by an arrow 706. The receiving portion 704 is rotatable about a point 704A in a direction indicated by an arrow 708. The receiving portion 704 receives the detent portion 702 at a first equilibrium location, a groove 710, as well as other locations away from the groove 710, such as an unstable location 712 and a second equilibrium location 714. FIG. 11B illustrates the detent assembly 700 at a first equilibrium location, with the detent portion 702 stably resting within the groove 710. FIG. 11C illustrates the detent assembly 700 at the unstable location 712. FIG. 11D illustrates the detent assembly 700 at the second equilibrium location 714. The detection mechanism (e.g., the detection mechanism 650) indicates a first state at the first equilibrium location 710 and a second state at the second equilibrium location 714. The second equilibrium location 714 can change enough so that a tracker arm mounted to the detent assembly moves sufficiently to indicate a detectable degradation.

A constant torque or force is induced on the detent portion 702 such that a sufficient force (or torque) must be applied to cause the receiving portion 704 to rotate in the direction indicated by the arrow 708 against the force on the detent portion 702 to cause the detent portion 702 to move from the first equilibrium location to another equilibrium location. While FIGS. 11B-11D depict the detent assembly 700 with a pawl detent portion 702 such that it rotates in the direction indicated by the arrow 706, other types of detent assemblies can be used. For example, a ball detent can be used in which a ball is spring-loaded to create a first equilibrium location when resting in the groove 710. Because the ball is spring-loaded, a sufficient force or torque must be applied to the receiving portion 704 in the direction indicated by the arrow 708 to cause the receiving portion 704 to rotate against the force from the spring so the ball moves from the equilibrium location within the groove 710 to another equilibrium location.

The detent assembly 650 is used by the detection mechanism 650 to create a plurality of equilibrium locations of the tracker arms 654, 656. Detents can be used to respond to forces about the tracker arm axes (e.g., the axes 666 and 668 for the tracker arm 654). With reference to FIGS. 11B-11D, the detent assemblies 700 are used to provide an initial state for the tracker arms 654, 656 of the detection mechanism 650 as shown in FIG. 11A, with the detent assemblies at the equilibrium location illustrated in FIG. 11B. Because a constant force is applied to the receiving portion 704 while the detent portion 702 is resting in the groove 710, a force must be induced to the tracker arms 654 or 656 (e.g., the markers 660A, 660B and/or the supports 654A-654D of the tracker arm 654) sufficient to move the receiving portion 704 against the torque of the detent portion 702 to cause the detent portion 702 to move to a different equilibrium location. A sufficient force induced on portions of either of the tracker arms 654, 656 changes the equilibrium location (or state) of the detent assembly along one or more of the axes 666, 668 or 670 (e.g., from the first equilibrium location within the groove 710 of FIG. 11B to the second equilibrium location 714 of FIG. 11D).

Trackers (e.g., the tracker 652) often require a particular geometry for the arrangement of the markers (e.g., the markers 660A-660B and 662A-662B) for proper recognition by a tracking system. For example, the geometric requirements can be based on the length between the markers, an angle between various combinations of three markers, and other three-dimensional spatial relationships. Because the tracker arms 654 and 656 are mounted using detent assemblies, forces applied to the tracker 652 that could affect registration can be monitored with the detent assemblies. If a sufficient force is induced on one or more of the tracker arms 654, 656, then one or more of the detent assemblie(s) will move to the second equilibrium location. FIG. 11E illustrates an exemplary depiction of the detection mechanism 650 at an invalid equilibrium location. The tracker arm 654 of FIG. 11A was subject to a sufficient force to cause it to rotate about the axis 666 (e.g., in a direction away from a plane created by the axes 666 and 668) to a location 654′. By moving from the first equilibrium location at 654 (e.g., with the detent assembly for the axis 666 at the equilibrium location of FIG. 11B) to a second equilibrium location at 654′ (e.g., with the detent assembly for the axis 666 at the equilibrium location of FIG. 11D), the geometric arrangement of the markers 660A, 660B, 662A and 662B is disturbed. A tracking system (e.g., the tracking system 40 of FIG. 1) can no longer recognize the tracker 652, which indicates a degradation of pose to the tracking system.

The initial equilibrium location (e.g., the equilibrium location of FIG. 11B) can be designed so a small angular change of the tracker arms causes the detent assembly to change from a stable equilibrium location to an unstable equilibrium location. By making the angular change of stability small at the first equilibrium location, small forces to the tracker can be monitored to granularly monitor for a degradation of pose. The first state of the tracker (e.g., the tracker arm 654) indicates no degradation of pose, and the second state of the tracker (e.g., the tracker arm 654′) indicates a degradation of pose. The second state of the tracker can be controlled so the tracker arms are only allowed to move a predetermined distance (e.g., to prevent the tracker arms from falling onto a rigid body or a patient). After a force causes the tracker to acquire a second equilibrium state, the components which moved can be restored to the original equilibrium location to achieve the same relative pose to the rigid body as when initially configured (e.g., from the location of the tracker arm 654′ of FIG. 11E to the location of the tracker arm 654 of FIG. 11A).

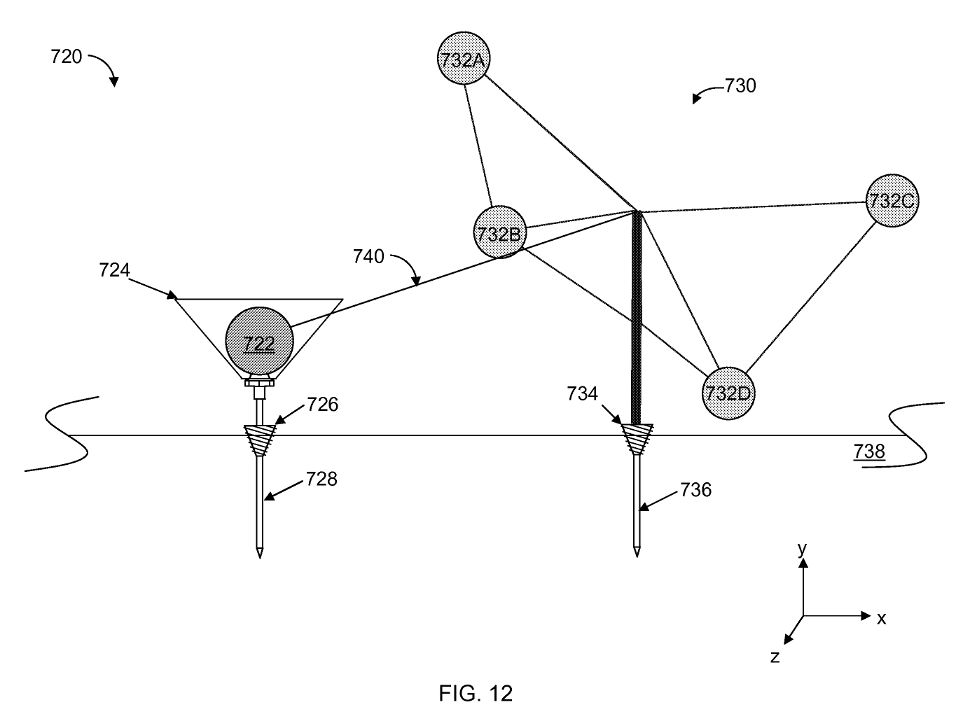

As described in FIGS. 11A-11E, the geometric relationship of markers on an individual tracker can be monitored for an indication of a degradation of pose. FIG. 12 illustrates another exemplary embodiment of a detection mechanism 720 of a self-detecting kinematic clamp assembly. The detection mechanism 720 includes a marker 722 protected by a soil shield 724. The marker 722 is mounted to a mounting device 726. In this embodiment, the mounting device 726 is a conically shaped object with spiral threads extending about the outer surface of the mounting device 726. For example, the mounting device 726 can be screwed into a rigid body 738 (e.g., the outer cortical shell of a bone) to constrain movement of the marker 722. The mounting device 726 includes a penetrating member 728 for providing additional support to the marker 722. The detection mechanism 720 also includes a tracker 730 with markers 732A-732D. In this embodiment, the tracker 730 is mounted to a mounting device 734 which can be screwed into the rigid body 738, and includes a penetrating member 736 for providing additional support. In some embodiments, the tracker 730 and rigid body 738 are connected via a self-detecting kinematic clamp assembly (e.g., the self-detecting kinematic clamp assembly 100 of FIG. 1).

As illustrated in FIG. 12, the marker 722 is mounted separately from the tracker 730. Upon registration by a tracking system of the rigid body 738, the initial relative relationship between the marker 722 and the markers 732A-D of the tracker 730 in three dimensional space (e.g., across the x, y, and z axes), as indicated by a line 740, is known and comprises a valid registration state. The relative relationship of the markers 722 and 732A-D can be monitored by the tracking system, and the locations of the markers during the valid registration state can be compared to the current monitored locations of the markers. A change in the relative relationship 740 is indicative of a degradation in the registration of the rigid body 738. Advantageously, because the marker 722 and the tracker 730 are each independently mounted (i.e., they are not attached to a common mounting structure), registration degradation caused by a loosening of a mounting device (e.g., the mounting device 734 or 736) can be detected.

A tracking system can monitor the marker 722 along three degrees of freedom (3DOF), namely movement along the x, y, and z axes (e.g., forward/backward, up/down, and left/right). Because the tracker 730 includes multiple markers in a known geometrical relationship, a tracking system can monitor the tracker 730 along six degrees of freedom (6DOF), namely movement along and about the x, y, and z axes (e.g., forward/backward, up/down, left/right, combined with rotation about the three axes (roll, yaw, pitch)). In some embodiments, the tracker 730 can be configured to monitor mDOF, where m>6. While FIG. 12 illustrates a single marker 722 to measure 3DOF, any type of tracking array or arrangement of markers can be used. For example, the “first tracker” (e.g., the single marker 722) can be a tracker with multiple markers (e.g., the tracker 730) to monitor 6DOF (e.g., when there are no workspace or mounting location restrictions). In some examples, if the first tracker has mDOF, where m≧3, the relative spatial position of the first tracker can be determined and monitored for any change relative to the validated registration state. In some examples, if the first tracker has mDOF, where m<3, some transducer motions of the first tracker may not be detected but also may not be of consequence if motions that do not include one of the mDOF motion components is not likely.

While FIG. 12 illustrates the marker 722 and the tracker 730 connected directly to the rigid body 738, the marker 722 and/or the tracker 730 need not be directly connected to the rigid body 738. For example, the marker 722 and/or the tracker 730 can be mounted on a kinematic clamp assembly as described above with reference to FIGS. 7A-7C. In some examples, the marker 722 and/or the tracker 730 are adjustable trackers as described in U.S. Pat. Application No. 2004/0068263, entitled “CAS Bone Reference with Articulated Support,” which is hereby incorporated by reference herein in its entirety. In some embodiments, the marker 722 and the tracker 730 are mounted on a common rigid body mount attached to the rigid body. Motion between the rigid body mount and the marker 722 and the tracker 730 can be detected and used to determine a degradation in registration. In some examples, the detection mechanism 720 is mounted on a non-rigid body. Full detection of movement of the non-rigid body can be detected when the first tracker has mDOF, where m≧6. Some detection of movement can be detected when the first tracker has mDOF, where m≧3, but some non-rigid phenomena may not be detected.

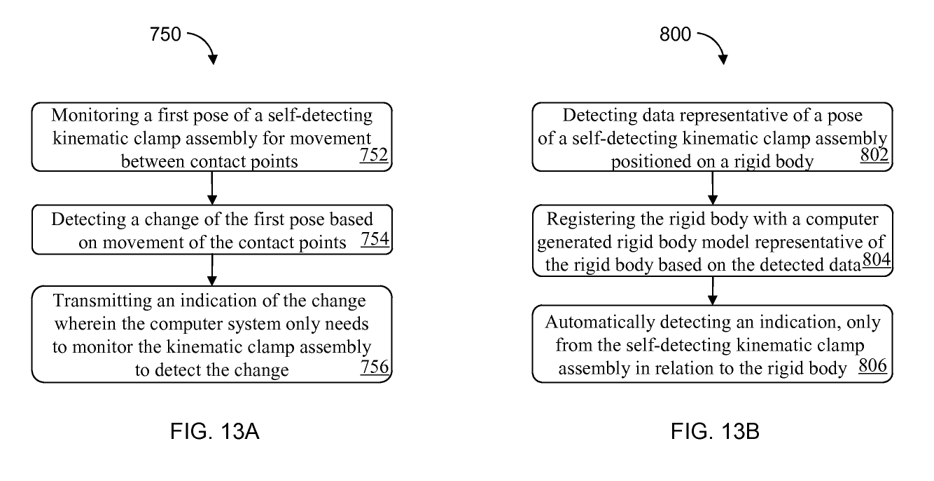

FIGS. 13A-13B illustrate computer implemented methods 750, 800 for automatically detecting registration degradation. Method 750 includes monitoring (752), by a self-detecting kinematic clamp assembly (e.g., the self-detecting kinematic clamp assembly 100 of FIG. 3) positioned on a rigid body, a first pose of the self-detecting kinematic clamp assembly (e.g., a pose of the self-detecting kinematic clamp assembly 100 with the base portion 302 kinematically assembled to the top portion 304) for movement between contact points between two portions of the self-detecting kinematic clamp assembly (e.g., for a separation between one or more of the balls 320 of FIG. 7A from a corresponding groove 316). The contact points can be monitored by a detection mechanism of the self-detecting kinematic clamp assembly (e.g., the circuit 353 of FIG. 8). The detection mechanism detects (754) a change of the first pose of the self-detecting kinematic clamp assembly based on movement of the contact points (e.g., a separation between one or more of the balls 320 from a corresponding groove 316). The self-detecting kinematic clamp assembly transmits (756) an indication of the change to a computer system (e.g., via a state indicator, such as the latch 354 or the optical transmitter 384 of FIG. 8) wherein the computer system only needs to monitor the self-detecting kinematic clamp assembly to detect the indication.

Method 800 includes a computer system (e.g., the computer system 10 of FIG. 1) detecting (802) data representative of a pose of a self-detecting kinematic clamp assembly positioned on a rigid body (e.g., via the detection device 41 of the tracking system 40 of FIG. 1). The computer system registers (804) the rigid body with a computer generated rigid body model representative of the rigid body based on the detected data. For example, utilizing pose data from the tracking system 40 of FIG. 1, the surgical system 10 is able to associate the physical anatomy (i.e., physical space) with a representation of the anatomy (i.e., image space). The computer system automatically detects (806) an indication, only from the self-detecting kinematic clamp assembly, representative of a change in the pose of the self-detecting kinematic clamp assembly in relation to the rigid body, wherein the change degrades the registration of the rigid body with the computer generated rigid body model (e.g., data representative of a change from a first state of the self-detecting kinematic clamp assembly to a second state, which is transmitted via a state indicator, such as the latch 354 or the optical transmitter 384 of FIG. 8). By monitoring the indication, the computer system can be alerted to poor integrity (e.g., sufficient to cause a registration degradation) of the self-detecting kinematic clamp assembly.

The above-described techniques can be implemented in digital and/or analog electronic circuitry, or in computer hardware, firmware, software, or in combinations of them. The implementation can be as a computer program product, i.e., a computer program tangibly embodied in a machine-readable storage device, for execution by, or to control the operation of, a data processing apparatus, e.g., a programmable processor, a computer, and/or multiple computers. A computer program can be written in any form of computer or programming language, including source code, compiled code, interpreted code and/or machine code, and the computer program can be deployed in any form, including as a stand-alone program or as a subroutine, element, or other unit suitable for use in a computing environment. A computer program can be deployed to be executed on one computer or on multiple computers at one or more sites.

Method steps can be performed by one or more processors executing a computer program to perform functions of the invention by operating on input data and/or generating output data. Method steps can also be performed by, and an apparatus can be implemented as, special purpose logic circuitry, e.g., a FPGA (field programmable gate array), a FPAA (field-programmable analog array), a CPLD (complex programmable logic device), a PSoC (Programmable System-on-Chip), ASIP (application-specific instruction-set processor), or an ASIC (application-specific integrated circuit). Subroutines can refer to portions of the computer program and/or the processor/special circuitry that implement one or more functions.

Processors suitable for the execution of a computer program include, by way of example, both general and special purpose microprocessors, and any one or more processors of any kind of digital or analog computer. Generally, a processor receives instructions and data from a read-only memory or a random access memory or both. The essential elements of a computer are a processor for executing instructions and one or more memory devices for storing instructions and/or data. Memory devices, such as a cache, can be used to temporarily store data. Memory devices can also be used for long-term data storage. Generally, a computer also includes, or is operatively coupled to receive data from or transfer data to, or both, one or more mass storage devices for storing data, e.g., magnetic, magneto-optical disks, or optical disks. A computer can also be operatively coupled to a communications network in order to receive instructions and/or data from the network and/or to transfer instructions and/or data to the network. Information carriers suitable for embodying computer program instructions and data include all forms of volatile and non-volatile memory, including by way of example semiconductor memory devices, e.g., DRAM, SRAM, EPROM, EEPROM, and flash memory devices; magnetic disks, e.g., internal hard disks or removable disks; magneto-optical disks; and optical disks, e.g., CD, DVD, HD-DVD, and Blu-ray disks. The processor and the memory can be supplemented by and/or incorporated in special purpose logic circuitry.

To provide for interaction with a user, the above described techniques can be implemented on a computer in communication with a display device, e.g., a CRT (cathode ray tube), plasma, or LCD (liquid crystal display) monitor, for displaying information to the user and a keyboard and a pointing device, e.g., a mouse, a trackball, a touchpad, or a motion sensor, by which the user can provide input to the computer (e.g., interact with a user interface element). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, and/or tactile input.

The above described techniques can be implemented in a distributed computing system that includes a back-end component. The back-end component can, for example, be a data server, a middleware component, and/or an application server. The above described techniques can be implemented in a distributed computing system that includes a front-end component. The front-end component can, for example, be a client computer having a graphical user interface, a Web browser through which a user can interact with an example implementation, and/or other graphical user interfaces for a transmitting device. The above described techniques can be implemented in a distributed computing system that includes any combination of such back-end, middleware, or front-end components.

The computing system can include clients and servers. A client and a server are generally remote from each other and typically interact through a communication network. The relationship of client and server arises by virtue of computer programs running on the respective computers and having a client-server relationship to each other.

The components of the computing system can be interconnected by any form or medium of digital or analog data communication (e.g., a communication network). Examples of communication networks include circuit-based and packet-based networks. Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), 802.11 network, 802.16 network, general packet radio service (GPRS) network, HiperLAN), and/or other packet-based networks. Circuit-based networks can include, for example, the public switched telephone network (PSTN), a private branch exchange (PBX), a wireless network (e.g., RAN, bluetooth, code-division multiple access (CDMA) network, time division multiple access (TDMA) network, global system for mobile communications (GSM) network), and/or other circuit-based networks.

Devices of the computing system can include, for example, a computer, a computer with a browser device, a telephone, an IP phone, a mobile device (e.g., cellular phone, personal digital assistant (PDA) device, laptop computer, electronic mail device), and/or other communication devices. The browser device includes, for example, a computer (e.g., desktop computer, laptop computer) with a world wide web browser (e.g., Microsoft® Internet Explorer® available from Microsoft Corporation, Mozilla® Firefox available from Mozilla Corporation). Mobile computing device include, for example, a Blackberry®. IP phones include, for example, a Cisco® Unified IP Phone 7985G available from Cisco System, Inc, and/or a Cisco® Unified Wireless Phone 7920 available from Cisco System, Inc.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

What is claimed is:

1. A self-detecting kinematic clamp assembly to detect and indicate a degradation in pose comprising:
a tracking element for tracking a pose of the kinematic clamp assembly;
a base portion comprising a fixation device that is attachable to a rigid body;
a top portion connected to the tracking element, the top portion being removably connected to the base portion; and
a detection mechanism comprising:
three or more contact points between the base potion and top portion; and
a mechanism that detects movement between one or more of the three or more contact points.

2. The kinematic clamp assembly of claim 1 wherein the tracking element comprises at least one of a marker and an array of markers.

3. The kinematic clamp assembly of claim 1 comprising six contact points arranged so that the detection mechanism can monitor six degrees of freedom of the kinematic clamp assembly.

4. The kinematic clamp assembly of claim 1 wherein the top portion is adjustably connected to the tracking element.

5. The kinematic clamp assembly of claim 4 further comprising a clamp that spherically connects the top portion to the tracking element to allow motion of the tracking element in any axis.

6. The kinematic clamp assembly of claim 5 wherein the clamp comprises:
a central member;
a first clamp arm hingedly connected to the central member so that the central member and the first clamp arm define a first adjustable spherical receptor; and
a tightening mechanism that extends through the first clamp arm and the central member to tighten the first adjustable spherical receptor.

7. The kinematic clamp assembly of claim 6 wherein:
the central member comprises a first end, a central area, and a second end; and
the first clamp arm is disposed along a top surface of the central member, wherein:
a first end of the first clamp arm is hingedly connected to the first end of the central member;
a second end of the first clamp arm is disposed at the central area of the central member; and
the top surface of the central member comprising a portion defining a first side of the first adjustable spherical receptor and a bottom surface of the first clamp arm comprising a portion defining a second side of the first adjustable spherical receptor.

8. The kinematic clamp assembly of claim 7 wherein the clamp further comprises a second clamp arm disposed along a bottom surface of the central member, wherein:
a first end of the second clamp arm is hingedly connected to the second end of the central member;
a second end of the second clamp arm is disposed at the central area of the central member; and
the bottom surface of the central member and a top surface of the first clamp arm define a second adjustable spherical receptor.

9. The kinematic clamp assembly of claim 8 wherein the tightening mechanism extends through the second end of the second clamp arm and the central area of the central member, and terminates at the second end of the first clamp arm.

10. The kinematic clamp assembly of claim 5 wherein the clamp comprises:
a first clamp side hingedly connected to a second clamp side so that the first clamp side and the second clamp side define a first adjustable spherical receptor; and
a tightening mechanism that extends through the first clamp side and terminates at the second clamp side to tighten the first adjustable spherical receptor.

11. The kinematic clamp assembly of claim 10 wherein the first adjustable spherical receptor comprises a lip that protrudes into a space defined by the first adjustable spherical receptor, wherein the lip extends about an inner portion of a top edge of the first adjustable spherical receptor.

12. The kinematic clamp assembly of claim 10 wherein the first clamp side and the second clamp side define a second adjustable spherical receptor, wherein the first adjustable spherical receptor is disposed on a bottom portion of the clamp and the second adjustable spherical receptor is disposed on a top portion of the clamp.

13. The kinematic clamp assembly of claim 1 wherein the base portion, the top portion, or both comprise one or more magnets.

14. The kinematic clamp assembly of claim 1 further comprising a flexible connector connected to the base portion and the top portion.

15. The kinematic clamp assembly of claim 1 wherein the top portion is removably connected to the base portion using a Maxwell Mount, a Kelvin Mount, a Canoe Ball/Vee Groove Mount, a Three Tooth Coupling, or any combination thereof.

16. The kinematic clamp assembly of claim 1 wherein the detection mechanism comprises a state indicator that indicates a change in pose based on the detection mechanism.

17. The kinematic clamp assembly of claim 16 wherein the detection mechanism comprises a circuit that drives the state indicator to a first state when each of the three or more contact points are in a first position and a second state based on movement of one or more of the three or more contact points.

18. The kinematic clamp assembly of claim 19 wherein the circuit drives the state indicator to the second state based on a separation of one or more of the three or more contact points.

19. The kinematic clamp assembly of claim 17 wherein the three or more contact points comprise an electrically conductive material.

20. The kinematic clamp assembly of claim 17 wherein the circuit includes a latching logical circuit.

21. The kinematic clamp assembly of claim 17 wherein the first state is indicated by a logical high and the second state is indicated by a logical low or the first state is indicated by a logical low and the second state is indicated by a logical high.

22. The kinematic clamp assembly of claim 17 wherein the state indicator comprises an optical transmitter for transmitting a signal representative of the first state, the second state, or both.

23. The kinematic clamp assembly of claim 22 wherein the optical transmitter comprises an LED, IR LCD shutter, or any combination thereof.

24. The kinematic clamp assembly of claim 1 wherein at least one of the three or more contact points comprises a detent assembly, the detent assembly comprising:
a detent portion; and
a receiving portion that receives the detent portion at least two locations.

25. The kinematic clamp assembly of claim 24 wherein an equilibrium at a first location of the at least two locations is less stable than an equilibrium at a second location of the at least two locations.

26. The kinematic clamp assembly of claim 25 wherein the state indicator indicates a first state at the first location and a second state at the second location.

27. The kinematic clamp assembly of claim 25 wherein the second location is located at a location at which a position of the tracking element changes to a detectable degradation.

28. The kinematic clamp assembly of claim 25 wherein the receiving mechanism rotates about an axis such that a force applied to the receiving mechanism causes the receiving mechanism to rotate against a force of the detent portion which moves the detent portion from the first location to the second location.

29. The kinematic clamp assembly of claim 1 wherein each of the three or more contact points comprises a load sensor for measuring a load value.

30. The kinematic clamp assembly of claim 29 further comprising an indicator to indicate a change in the measured load value.

31. The kinematic clamp assembly of claim 29 wherein the load sensor comprises a strain gauge.

32. The kinematic clamp assembly of claim 29 further comprising a circuit, the circuit comprising, for each load sensor, a strain comparator, wherein:
the circuit drives a state indicator to a first state when each of the strain comparators indicates a preload value within a predetermined maximum preload and a predetermined minimum preload; and
the circuit drives the state indicator to a second state if one or more of the strain comparators indicates a preload value not within the predetermined maximum preload and the predetermined minimum preload.

33. A computer implemented method for automatically detecting registration degradation, comprising:
monitoring, by a self-detecting kinematic clamp assembly positioned on a rigid body, a first pose of the self-detecting kinematic clamp assembly for movement between contact points between two portions of the self-detecting kinematic clamp assembly;
detecting a change of the first pose of the self-detecting kinematic clamp assembly based on movement of the contact points; and
transmitting an indication of the change to a computer system wherein the computer system only needs to monitor the self-detecting kinematic clamp assembly to detect the indication.

34. A computer implemented method for automatically detecting registration degradation, comprising:
detecting data representative of a pose of a self-detecting kinematic clamp assembly positioned on a rigid body;
registering the rigid body with a computer generated rigid body model representative of the rigid body based on the detected data; and
automatically detecting an indication, only from the self-detecting kinematic clamp assembly, representative of a change in the pose of the self-detecting kinematic clamp assembly in relation to the rigid body, wherein the change degrades the registration of the rigid body with the computer generated rigid body model.
